(19) United States
(12) Patent Application Publication
(10) Pub. No.: US 2021/0030141 A1
(43) Pub. Date: Feb. 4, 2021

(54) MASK, DEVICE, AND METHOD FOR WHITENING SKIN

(71) Applicant: Boncheol GOO, Seoul (KR)

(72) Inventor: Boncheol GOO, Seoul (KR)

(21) Appl. No.: 16/856,950

(22) Filed: Apr. 23, 2020

Related U.S. Application Data
(60) Provisional application No. 62/880,541, filed on Jul. 30, 2019.

(30) Foreign Application Priority Data
Jan. 3, 2020 (KR) 10-2020-0001054
Jan. 3, 2020 (KR) 10-2020-0001055
Jan. 3, 2020 (KR) 10-2020-0001056

(51) Int. Cl. A45D 44/00; A61B 18/02

(52) U.S. Cl. CPC A45D 44/002; A61B 2018/00791; A61B 2018/0237; A61B 18/02

(57) ABSTRACT

The present disclosure relates to a mask, a device and a method for whitening skin. In an embodiment, the mask includes a contact layer having a lower portion configured to contact a face surface of a user, and at least one thermoelectric module including a first major surface disposed in contact with an upper portion of the contact layer and a second major surface positioned opposite to the first major surface. The mask also includes a heat dissipation layer being disposed in contact with the second major surface of the thermoelectric module. The mask further includes a controller configured to control the thermoelectric module to: cool the first major surface at a cooling temperature such that the skin of the user reaches a target temperature at which a pigmentation by melanocytes on the user's skin is suppressed, and maintain the cooling of the first major surface for a cooling interval.

### CROSS-REFERENCE TO RELATED APPLICATIONS

This application claims priority to U.S. Provisional Application No. 62/880,541 filed on Jul. 30, 2019, the disclosure of which is incorporated herein by reference in its entirety.

This application also claims priority to Korean Patent Application Nos. 10-2020-0001054, 10-2020-0001055 and 10-2020-0001056 all filed on Jan. 3, 2020 under 35 U.S.C. § 119, the disclosures of which are incorporated by reference in their entirety.

### BACKGROUND

### 1. Technical Field

The present disclosure relates to a mask, a device, and a method for whitening skin, and more particularly, to a mask, a device and a method for whitening skin that are capable of whitening the skin using cryotherapy.

### 2. Discussion of Related Technology

In recent years, with the improvement in the quality of life, interest in skin care has been increased. In skin care, there are demands for mole removal, pore size reduction, scar removal, wrinkle reduction, and the like, but there is a great demand for skin whitening that lightens the overall skin tone.

To this end, conventionally, skin whitening has been performed by absorbing functional materials, which act to inhibit melanin-producing enzymes in order to lighten the skin tone, into the skin. However, the functional materials have problems in that, due to safety problems such as causing irritation or rash when applied to skin, a use amount is limited, or, due to an insignificant effect, it is substantially difficult to expect the skin whitening effect to occur.

Therefore, there is a need to develop a technology that substantially performs skin whitening.

### SUMMARY

The present disclosure is directed to providing a mask, a device, and a method for whitening skin capable of transferring negative heat, which is generated according to an endothermic reaction of a thermoelectric module, without change to a face, thereby conveniently and effectively cooling facial skin of a user and obtaining a whitening effect.

The present disclosure is also directed to providing a mask, a device, and a method for whitening skin capable of controlling a temperature of a cooling surface of a thermoelectric module and an interval during which the skin is cooled by the thermoelectric module, thereby obtaining an effect of preventing damage to the skin.

The present disclosure is also directed to providing a mask, a device, and a method for whitening skin capable of maintaining a heat generating surface of a thermoelectric module at a predetermined temperature, thereby obtaining an effect of allowing smooth operation of the thermoelectric module.

The present disclosure is also directed to providing a mask, a device, and a method for whitening skin capable of controlling a temperature of a thermoelectric module to be within a temperature range in which a functional material is active, thereby obtaining an effect of activating the functional material.

The present disclosure is also directed to providing a mask, a device, and a method for whitening skin capable of controlling a temperature of a thermoelectric module to a temperature for obtaining additional skin improvement effects such as lipolysis and reduction of swelling, thereby obtaining the additional skin improvement effects.

Objectives of the present disclosure are not limited to those described above, and other unmentioned objectives should be clearly understood by those of ordinary skill in the art to which the present disclosure pertains from the present specification and the accompanying drawings.

According to one aspect of the present specification, there is provided a mask for whitening skin, the mask comprising a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer being disposed in contact with the second major surface of the thermoelectric module, wherein the heat dissipation layer receives, via the second major surface, a heat generated upon the cooling of the first major surface and dissipates the generated heat and a controller for controlling the thermoelectric module to: cool the first major surface at a cooling temperature such that the skin of the user reaches a target temperature at which a pigmentation by melanocytes on the user's skin is suppressed, wherein the target temperature is in the range of 4° C. to 27° C. and the cooling temperature is in the range of −15° C. to 15° C. and lower than the target temperature, and maintain the cooling of the first major surface for a cooling interval, wherein the cooling interval is in range of a minimum interval necessary for suppressing the pigmentation and a maximum interval for preventing a skin damage, the minimum interval being greater than 5 seconds and the maximum interval being smaller than 300 seconds.

According to another aspect of the present specification, there is provided a mask for whitening skin, the mask comprising a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a fluid circulation unit for receiving, via the second major surface, a heat generated upon the cooling of the first major surface and exchanging heat which is received, the fluid circulation unit including: a fluid chamber being disposed in contact with on the thermoelectric module to contact the second major surface of the thermoelectric module and being connected to a fluid inlet passage and a fluid outlet passage, a fluid tank for storing a fluid supplied, via the fluid inlet passage, to the fluid chamber, a pump for supplying, via the fluid inlet passage, the fluid stored in the fluid tank to the fluid chamber and retrieving, via the fluid outlet passage, the fluid to the fluid tank by pumping, and a cooler for cooling the fluid retrieved from the fluid outlet passage, and, a controller for controlling the thermoelectric module to: cool the first major surface at a cooling temperature such that the skin of the user reaches a target temperature at which a pigmentation by melanocytes on the user's skin is suppressed, wherein the target temperature is in the range of 4° C. to 27° C. and the cooling temperature is in the range of −15° C. to 15° C. and lower than the target temperature, and maintain the cooling of the first major surface for a cooling interval, wherein the cooling interval is in range of a minimum interval necessary for suppressing the pigmentation and a maximum interval for preventing a skin damage, the minimum interval being greater than 5 seconds and the maximum interval being smaller than 300 seconds.

According to another aspect of the present specification, there is provided a device for whitening skin, the device comprising a contact layer having a lower portion being contacted with a body surface of a user, and provided as a flexible material for a tight-contact between the lower portion and the body surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer being disposed in contact with the second major surface of the thermoelectric module, wherein the heat dissipation layer receives, via the second major surface, a heat generated upon the cooling of the first major surface and dissipates the generated heat and a controller for controlling the thermoelectric module to: cool the first major surface at a cooling temperature such that the skin of the user reaches a target temperature at which a pigmentation by melanocytes on the user's skin is suppressed, wherein the target temperature is in the range of 4° C. to 27° C. and the cooling temperature is in the range of −15° C. to 15° C. and lower than the target temperature, and maintain the cooling of the first major surface for a cooling interval, wherein the cooling interval is in range of a minimum interval necessary for suppressing the pigmentation and a maximum interval for preventing a skin damage, the minimum interval being greater than 5 seconds and the maximum interval being smaller than 300 seconds.

According to another aspect of the present specification, there is provided a method for whitening skin, the method comprising, providing a mask onto a face surface of a user so that the mask is contacted with the face surface tightly, wherein the mask comprises a contact layer having a lower portion configured to be contacted with the face surface of the user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, a heat dissipation layer being disposed in contact with the second major surface of the thermoelectric module, and a controller for controlling the thermoelectric module, wherein the thermoelectric module cools the first major surface and applies, via the contact layer, a negative heat to a skin of the user, and wherein the heat dissipation layer receives, via the second major surface, a heat generated upon the cooling of the first major surface and dissipates the generated heat, applying the negative heat, via the contact layer, to the skin of the user by cooling the first major surface, suppressing a pigmentation by melanocytes on the user's skin by cooling the first major surface at a cooling temperature such that the skin of the user reaches a target temperature, wherein the target temperature is in the range of 4° C. to 27° C. and the cooling temperature is in the range of −15° C. to 15° C. and lower than the target temperature and preventing a skin damage by maintaining the cooling of the first major surface for a cooling interval, wherein the cooling interval is in range of a minimum interval necessary for suppressing the pigmentation and a maximum interval for preventing the skin damage, the minimum interval being greater than 5 seconds and the maximum interval being smaller than 300 seconds.

Means for achieving objectives of the present invention are not limited to those described above, and other unmentioned means should be clearly understood by those of ordinary skill in the art to which the present invention pertains from the present specification and the accompanying drawings.

### BRIEF DESCRIPTION OF THE DRAWINGS

The above and other objects, features and advantages of the described technology will become more apparent to those of ordinary skill in the art by describing embodiments thereof in detail with reference to the accompanying drawings.

FIG. 1 is a view illustrating a state in which a user uses a skin whitening mask according to an embodiment of the present specification.

FIG. 2 is an exploded perspective view of the skin whitening mask according to an embodiment of the present specification.

FIG. 3 is a view illustrating a contact layer at which an identification tag is disposed according to an embodiment of the present specification.

FIG. 4 is a view illustrating thermoelectric modules disposed inside a mask according to an embodiment of the present specification.

FIG. 5 illustrates a state in which negative heat of the mask is transferred to the skin according to an embodiment of the present specification.

FIG. 6 illustrates a cross-sectional view of a mask including a phase-change material heat dissipation layer that has a heat transfer member according to an embodiment of the present specification.

FIG. 7 illustrates a cross-sectional view of a mask including a metal heat dissipation layer according to an embodiment of the present specification.

FIG. 8 illustrates a cross-sectional view of a mask including a fan according to an embodiment of the present specification.

FIG. 9 is a block diagram of a controller according to an embodiment of the present specification.

FIG. 10 is a graph showing temperature changes over time of a first major surface of a thermoelectric module according to a first embodiment of the present specification.

FIG. 11 is a graph showing temperature changes over time of a first major surface of a thermoelectric module according to a second embodiment of the present specification.

FIG. 12 is a graph showing temperature changes over time of a first major surface of a thermoelectric module according to a third embodiment of the present specification.

FIG. 13 is an example graph showing activity of a functional material according to temperature.

FIG. 14 is a graph showing temperature changes over time of a first major surface of a thermoelectric module according to a fourth embodiment of the present specification.

FIG. 15 is a graph showing temperature changes over time of a first major surface of a thermoelectric module according to a fifth embodiment of the present specification.

FIG. 16 is a graph showing temperature changes over time of a first major surface of a thermoelectric module according to a sixth embodiment of the present specification.

FIG. 17 illustrates a cradle on which a mask is mounted according to an embodiment of the present specification.

FIG. 18 is a perspective view of a skin whitening device according to an embodiment of the present specification.

FIG. 19 is a view illustrating a state in which a skin whitening device is used for an upper body part of a user according to an embodiment of the present specification.

FIG. 20 is a view illustrating a state in which a skin whitening device is used for a wrist area of a user according to an embodiment of the present specification.

FIG. 21 is a view illustrating a state in which a skin whitening device is used for a foot area of a user according to an embodiment of the present specification.

FIG. 22 is a flowchart of a skin whitening method using a skin whitening mask according to an embodiment of the present specification.

FIG. 23 is a flowchart of a skin whitening method using a skin whitening mask according to an embodiment of the present specification.

FIG. 24 illustrates a schematic exploded perspective view of a mask including a fluid circulation unit according to an embodiment of the present specification.

FIG. 25 illustrates a cross-sectional view of the mask including the fluid circulation unit according to an embodiment of the present specification.

### DETAILED DESCRIPTION OF EMBODIMENTS

In the drawings, the thicknesses of layers and regions are exaggerated for clarity. Also, when an element or layer is described as being “on” or “above” another element or layer, this includes both a case in which the element or layer is directly on the other element or layer and a case in which still another element or layer is interposed therebetween. In principle, like reference numerals refer to like elements throughout. Also, elements having the same function within the scope of the same idea shown in the drawings of each embodiment will be described using the same reference numerals.

When detailed description of known functions or configurations related to the present disclosure is deemed as having the possibility of unnecessarily blurring the gist of the described technology, the detailed description thereof will be omitted. Also, ordinals (e.g., first and second) used in the description process of the present specification are merely identification symbols for distinguishing one element from another element.

In addition, the terms “module” and “part” which are used to refer to elements in the following description have been given or used in combination with other terms only in consideration of ease of writing the specification and thus do not have meanings or roles that are distinguished from each other.

When a heat exchange occurs between objects with different temperatures, it may be assumed that an object with a relatively higher temperature generates heat and an object with a relatively lower temperature absorbs the heat. Therefore, when an arbitrary object is adjacent to the human skin, the arbitrary object may generate thermal energy when a temperature of the arbitrary object is higher than a temperature of the human skin, and the arbitrary object may absorb thermal energy when the temperature of the arbitrary object is lower than the temperature of the human skin. Thus, in the present specification, absorbing thermal energy from the human skin by the arbitrary object may refer to “applying negative heat to the human skin.” Also, the term “cooling” used herein may refer to lowering a temperature of an object to be cooled to a temperature lower than that before cooling the object.

In the present specification, “whitening skin” refers to any action that inhibits melanin synthesis and suppresses or prevents pigmentation due to melanin. For example, skin whitening may include lightening a skin tone as a result of cooling the skin and suppressing pigmentation of the skin, removing a mole on the skin, and the like.

In the present specification, an operation mode may refer to cooling a cooling surface of a thermoelectric module according to predetermined cooling temperature and cooling interval.

In the present specification, a whitening mode may refer to cooling the cooling surface of the thermoelectric module according to a predetermined cooling condition for causing a whitening effect of skin.

In the present specification, a functional material activation mode may refer to cooling the cooling surface of the thermoelectric module according to a predetermined cooling condition for causing an effect of activating a functional material.

In the present specification, a lipolysis mode may refer to cooling the cooling surface of the thermoelectric module according to a predetermined cooling condition for causing a lipolytic effect.

In the present specification, a swelling reduction mode may refer to cooling the cooling surface of the thermoelectric module according to a predetermined cooling condition for causing an effect of reducing swelling.

One aspect of the present specification may provide a mask for whitening skin, the mask comprising a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer being disposed in contact with the second major surface of the thermoelectric module, wherein the heat dissipation layer receives, via the second major surface, a heat generated upon the cooling of the first major surface and dissipates the generated heat and a controller for controlling the thermoelectric module to: cool the first major surface at a cooling temperature such that the skin of the user reaches a target temperature at which a pigmentation by melanocytes on the user's skin is suppressed, wherein the target temperature is in the range of 4° C. to 27° C. and the cooling temperature is in the range of −15° C. to 15° C. and lower than the target temperature, and maintain the cooling of the first major surface for a cooling interval wherein the cooling interval is in range of a minimum interval necessary for suppressing the pigmentation and a maximum interval for preventing a skin damage, the minimum interval being greater than 5 seconds and the maximum interval being smaller than 300 seconds.

In addition, the mask may further comprise a skin temperature sensor for measuring a skin temperature of the skin and wherein the controller controls the thermoelectric module based on the skin temperature measured from the skin temperature sensor.

In addition, the controller may control the thermoelectric module to maintain the measured temperature at the target temperature for 4 seconds to 120 seconds.

In addition, the mask may further comprise a vibration generating module for outputting vibration applied to the face surface; and wherein the controller controls the vibration generating module to output a vibration during or after the cooling of the first major surface.

In addition, the mask may further comprise a touch sensing module for sensing a contact of the skin with the mask and wherein the controller controls the thermoelectric module to start the cooling of the first major surface when the contact of the skin is detected by the touch sensing module.

In addition, the controller may control the thermoelectric module to slowly increase a temperature of the first major surface after the cooling of the first major surface.

In addition, the thermoelectric module may include a plurality of thermoelectric module groups, and each of the plurality of thermoelectric module groups is controlled separately by the controller.

In addition, the plurality of thermoelectric module groups may correspond to a plurality of face surface sections being defined based on a skin temperature profile.

In addition, the plurality of thermoelectric module groups may include a first thermoelectric group corresponding to a first section and a second thermoelectric group corresponding to a second section of which the skin temperature is lower than that of the first section, and the controller controls the first thermoelectric group to perform the cooling of the first major surface corresponding to the first section for a first cooling interval and the second thermoelectric group to perform the cooling of the first major surface corresponding to the second section for a second cooling interval, the first cooling interval being greater than the second cooling interval.

In addition, the plurality of thermoelectric module groups may include a first thermoelectric group corresponding to a first section and a second thermoelectric group corresponding to a second section of which the skin temperature is lower than that of the first section, and the controller controls the first thermoelectric group to perform the cooling of the first major surface corresponding to the first section at a first cooling temperature and the second thermoelectric group to perform the cooling of the first major surface corresponding to the second section at a second cooling temperature, the first cooling temperature being higher than the second cooling temperature.

In addition, the controller may further include the an input module for obtaining a cooling condition of the first major surface, and control the thermoelectric module to cool the first major surface according to the cooling condition inputted from the input module.

In addition, the controller may further include a communication module for communicating with an external device, and control the thermoelectric module to cool the first major surface according to the cooling condition obtained from the communication module.

In addition, the controller may control the thermoelectric module to cool the first surface according to a first cooling condition related to a swelling reduction effect of the skin or a second cooling condition related to lipolysis effect of the skin during at least a portion of the cooling interval of the first major surface.

In addition, the thermoelectric module may have flexibility.

In addition, the heat dissipation layer may include a fluid chamber which receives fluid from a fluid supply module storing the fluid, and exchanges heat by receiving the heat generated upon the cooling of the first major surface.

In addition, the fluid chamber may include a plurality of chambers, the plurality of chambers being connected in series and/or in parallel between the chambers.

In addition, another aspect of the present specification may provide a mask for whitening skin, the mask comprising a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a fluid circulation unit for receiving, via the second major surface, a heat generated upon the cooling of the first major surface and exchanging heat which is received, the fluid circulation unit including: a fluid chamber being disposed in contact with on the thermoelectric module to contact the second major surface of the thermoelectric module and being connected to a fluid inlet passage and a fluid outlet passage, a fluid tank for storing a fluid supplied, via the fluid inlet passage, to the fluid chamber, a pump for supplying, via the fluid inlet passage, the fluid stored in the fluid tank to the fluid chamber and retrieving, via the fluid outlet passage, the fluid to the fluid tank by pumping, and a cooler for cooling the fluid retrieved from the fluid outlet passage, and, a controller for controlling the thermoelectric module to: cool the first major surface at a cooling temperature such that the skin of the user reaches a target temperature at which a pigmentation by melanocytes on the user's skin is suppressed, wherein the target temperature is in the range of 4° C. to 27° C. and the cooling temperature is in the range of −15° C. to 15° C. and lower than the target temperature, and maintain the cooling of the first major surface for a cooling interval, wherein the cooling interval is in range of a minimum interval necessary for suppressing the pigmentation and a maximum interval for preventing a skin damage, the minimum interval being greater than 5 seconds and the maximum interval being smaller than 300 seconds.

In addition, another aspect of the present specification may provide a device for whitening skin, the device comprising a contact layer having a lower portion being contacted with a body surface of a user, and provided as a flexible material for a tight-contact between the lower portion and the body surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer being disposed in contact with the second major surface of the thermoelectric module, wherein the heat dissipation layer receives, via the second major surface, a heat generated upon the cooling of the first major surface and dissipates the generated heat and a controller for controlling the thermoelectric module to: cool the first major surface at a cooling temperature such that the skin of the user reaches a target temperature at which a pigmentation by melanocytes on the user's skin is suppressed, wherein the target temperature is in the range of 4° C. to 27° C. and the cooling temperature is in the range of −15° C. to 15° C. and lower than the target temperature, and maintain the cooling of the first major surface for a cooling interval, wherein the cooling interval is in range of a minimum interval necessary for suppressing the pigmentation and a maximum interval for preventing a skin damage, the minimum interval being greater than 5 seconds and the maximum interval being smaller than 300 seconds.

In addition, another aspect of the present specification may provide a method for whitening skin, the method comprising, providing a mask onto a face surface of a user so that the mask is contacted with the face surface tightly, wherein the mask comprises a contact layer having a lower portion configured to be contacted with the face surface of the user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, a heat dissipation layer being disposed in contact with the second major surface of the thermoelectric module, and a controller for controlling the thermoelectric module, wherein the thermoelectric module cools the first major surface and applies, via the contact layer, a negative heat to a skin of the user, and wherein the heat dissipation layer receives, via the second major surface, a heat generated upon the cooling of the first major surface and dissipates the generated heat, applying the negative heat, via the contact layer, to the skin of the user by cooling the first major surface, suppressing a pigmentation by melanocytes on the user's skin by cooling the first major surface at a cooling temperature such that the skin of the user reaches a target temperature, wherein the target temperature is in the range of 4° C. to 27° C. and the cooling temperature is in the range of −15° C. to 15° C. and lower than the target temperature and preventing a skin damage by maintaining the cooling of the first major surface for a cooling interval, wherein the cooling interval is in range of a minimum interval necessary for suppressing the pigmentation and a maximum interval for preventing the skin damage, the minimum interval being greater than 5 seconds and the maximum interval being smaller than 300 seconds.

In addition, another aspect of the present specification may provide a mask that for whitening skin, the mask including, a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer disposed at an upper portion of the thermoelectric module so as to come in contact with the second major surface of the thermoelectric module and configured to receive, via the second major surface, heat generated upon cooling of the first major surface and dissipate the received heat, and a controller configured to control the thermoelectric module to cool the first major surface within a whitening temperature range in which pigmentation by melanocytes is suppressed and control the thermoelectric module to cause the whitening temperature range and the activation temperature range of the functional material to overlap during at least a portion of a whitening interval, during which the cooling of the first major surface occurs within the whitening temperature range.

In addition, the functional material may be formed of at least one of an ingredient that helps protect against UV rays, an ingredient that prevents oxidation, an ingredient that conditions the skin, an ingredient that inhibits the action of bacteria, an ingredient that whitens the skin, an ingredient that reduces the size of pores, an ingredient that reduces wrinkles, an ingredient that revitalizes the skin, an ingredient that prevents burning sensation, and an ingredient that relieves pain or a mixture of two or more thereof.

In addition, the functional material may include a material whose skin improving function is further activated at low temperature.

In addition, the functional material may be a material whose whitening effect is further enhanced at low temperature and may be formed of at least one of resorcinol and similar derivatives hexyl resorcinol, butyl resorcinol, phenylethyl resorcinol, resorcinol acetate, and other similar derivatives) or a mixture of two or more thereof.

In addition, the functional material may be a material that further reduces irritation at low temperature and may be formed of at least one of, or a mixture of two or more of, niacinamide and a composition containing the same, magnesium ascorbylphosphate and a composition containing the same, ascorbyl glucoside and a composition containing the same, ascorbyl tetraisopalmitate/dipalmitate and a composition containing the same, arbutin and a composition containing the same, α-bisabolol and a composition containing the same, ethyl ascorbyl ether and a composition containing the same, polyphenol derivatives and a composition containing the same, L-glutathione and a composition containing the same, tranexamic acid and a composition containing the same, 4-methoxysalicylic acid potassium salt (KCl) derivatives and a composition containing the same, glycyrrhizine and a composition containing the same, azelaic acid, azelaic acid derivatives (e.g., azeloyl diglycine) and a composition containing the same, nicotinamide, nicotinamide derivatives and a composition containing the same, resveratrol, resveratrol derivatives and a composition containing the same, glycyrrhiza flavonoids, ellagic acid and a composition containing the same, papain and a composition containing the same, mandelic acid, mandelic acid derivatives and a composition containing the same, heptapeptide-1 and a composition containing the same, kojic acid, kojic acid derivatives and a composition containing the same, and plant extracts and a composition containing the same that contain all or some of the following ingredients: jasmine extract, mulberry extract, paper mulberry extract, licorice extract, ginseng extract, salvia miltiorrhiza extract, corn extract, chrysanthemum extract, bark root extract, thyme extract, white fresh root extract, polygon extract, magnolia tree extract, angelica root extract, phyllanthus emblica (fruit) extract, and citrus extract.

In addition, the controller may control the thermoelectric module to cool the first major surface at a temperature at which the activity of the functional material is maximized.

In addition, when the functional material is provided as a plurality of functional materials, the controller may control the thermoelectric module to cause the whitening temperature range and the activation temperature range of at least one of the plurality of functional materials to overlap.

In addition, the whitening temperature range may be a range of −15° C. to 15° C.

In addition, the whitening interval may be maintained between 5 seconds, which is necessary for suppressing the pigmentation, and less than 300 seconds, at which damage to the skin begins.

In addition, the contact layer may be separable from the mask.

In addition, the mask may further include an identification tag which is disposed at the contact layer and has identification data corresponding to the functional material and a tag recognition module configured to recognize the identification tag, and the controller may identify the functional material from the identification data obtained by the tag recognition module recognizing the identification tag.

In addition, the identification tag may have data relating to a cooling condition of the first major surface that corresponds to the identification data, and the controller may control the thermoelectric module to cool the first major surface according to the cooling condition of the first major surface corresponding to the identification data that is obtained by the tag recognition module recognizing the identification tag.

Another aspect of the present specification may provide a mask for whitening skin, the mask comprising, a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer disposed at an upper portion of the thermoelectric module so as to come in contact with the second major surface of the thermoelectric module and configured to receive, via the second major surface, heat generated upon cooling of the first major surface and dissipate the received heat, and a controller configured to control the thermoelectric module to perform a first mode in which the first major surface is cooled at a temperature at which pigmentation by melanocytes is suppressed in order to whiten the skin or a second mode in which the first major surface is cooled within an activation temperature range of the functional material in order to improve the activity of the functional material.

In addition, the functional material may be formed of at least one of an ingredient that helps protect against UV rays, an ingredient that prevents oxidation, an ingredient that conditions the skin, an ingredient that inhibits the action of bacteria, an ingredient that whitens the skin, an ingredient that reduces the size of pores, an ingredient that reduces wrinkles, an ingredient that revitalizes the skin, an ingredient that prevents burning sensation, and an ingredient that relieves pain or a mixture of two or more thereof.

In addition, the functional material may include a material whose skin improving function is further activated at low temperature.

In addition, the functional material may be a material whose whitening effect is further enhanced at low temperature and may be formed of at least one of resorcinol and similar derivatives (hexyl resorcinol, butyl resorcinol, phenylethyl resorcinol, resorcinol acetate, and other similar derivatives) or a mixture of two or more thereof.

In addition, the functional material may be a material that further reduces irritation at low temperature and may be formed of at least one of, or a mixture of two or more of, niacinamide and a composition containing the same, magnesium ascorbylphosphate and a composition containing the same, ascorbyl glucoside and a composition containing the same, ascorbyl tetraisopalmitate/dipalmitate and a composition containing the same, arbutin and a composition containing the same, α-bisabolol and a composition containing the same, ethyl ascorbyl ether and a composition containing the same, polyphenol derivatives and a composition containing the same, L-glutathione and a composition containing the same, tranexamic acid and a composition containing the same, 4-methoxysalicylic acid potassium salt (KCl) derivatives and a composition containing the same, glycyrrhizine and a composition containing the same, azelaic acid, azelaic acid derivatives (e.g., azeloyl diglycine) and a composition containing the same, nicotinamide, nicotinamide derivatives and a composition containing the same, resveratrol, resveratrol derivatives and a composition containing the same, glycyrrhiza flavonoids, ellagic acid and a composition containing the same, papain and a composition containing the same, mandelic acid, mandelic acid derivatives and a composition containing the same, heptapeptide-1 and a composition containing the same, kojic acid, kojic acid derivatives and a composition containing the same, and plant extracts and a composition containing the same that contain all or some of the following ingredients: jasmine extract, mulberry extract, paper mulberry extract, licorice extract, ginseng extract, salvia miltiorrhiza extract, corn extract, chrysanthemum extract, bark root extract, thyme extract, white fresh root extract, polygon extract, magnolia tree extract, angelica root extract, phyllanthus emblica (fruit) extract, and citrus extract.

In addition, the controller may control the thermoelectric module to maintain an interval during which the first mode is performed to be shorter than an interval during which the second mode is performed.

In addition, the controller may control the thermoelectric module to cool the first major surface at a higher temperature when performing the second mode as compared to when performing the first mode.

In addition, when the functional material is provided as a plurality of functional materials, the controller may control the thermoelectric module to cool the first major surface within the activation temperature range of at least one of the plurality of functional materials while performing the second mode.

In addition, the controller may control the thermoelectric module to perform the second mode simultaneously with the first mode during at least a portion of the interval during which the first mode is performed.

In addition, the first mode may be a mode in which the first major surface is cooled within a temperature range of −15° C. to 15° C.

In addition, the first mode may be a mode in Which the cooling of the first major surface is maintained between 5 seconds, which is necessary for suppressing the pigmentation, and less than 300 seconds, at which damage to the skin begins.

In addition, the second mode may be a mode in which the first major surface is cooled at a temperature at which the activity of the functional material is maximized.

In addition, another aspect of the present specification may provide a method for whitening skin, the method comprising, positioning onto a face of a user, a mask, which includes a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a heat dissipation layer disposed at an upper portion of the thermoelectric module so as to come in contact with the second major surface of the thermoelectric module and configured to receive, via the second major surface, heat generated upon cooling of the first major surface and dissipate the received heat, and a controller configured to control the thermoelectric module, so that the lower portion of the contact layer adheres to the face, as the power is applied to the thermoelectric module, cooling the first major surface and applying negative heat to the skin of the user via the contact layer, by the controller, performing whitening of the skin by cooling the first major surface within a whitening temperature range in which pigmentation by melanocytes is suppressed, and by the controller, activating the functional material by causing the whitening temperature range and the activation temperature range of the functional material to overlap during at least a portion of a whitening interval, during which the cooling of the first major surface occurs within the whitening temperature range.

In addition, another aspect of the present specification may provide a mask for whitening skin, the mask comprising, a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a phase-change material heat dissipation layer which is disposed at an upper portion of the thermoelectric module so as to come in contact with the second major surface of the thermoelectric module and includes a phase-change material which receives, via the second major surface, heat generated upon cooling of the first major surface and absorbs the received heat and maintains the temperature of the second major surface constant using latent heat at a melting point in order to maintain a difference between a temperature of the first major surface and a temperature of the second major surface to be less than a predetermined temperature, and a controller configured to control the thermoelectric module to cool the first major surface at a temperature at which pigmentation by melanocytes is suppressed in order to whiten the skin.

In addition, the predetermined temperature may be selected from a range of 30° C. to less than 55° C.

In addition, the phase-change material may have a melting point in a range of −15° C. to 40° C.

In addition, a mass of the phase-change material may be determined on the basis of input power and use time of the thermoelectric module, an amount of heat absorbed by the first major surface, and the latent heat at the melting point of the phase-change material.

In addition, a mass G of the phase-change material may be determined by Equation 1 below.

$$G = \frac{( {Q}_{c} + P ) \cdot t}{( Δ H )} \qquad [ Equation 1 ]$$

Here, Q_{C} represents the amount of heat absorbed by the first major surface, P represents the input power of the thermoelectric module, t represents the use time of the thermoelectric module, and ΔH represents the latent heat at the melting point of the phase-change material.

In addition, the phase-change material heat dissipation layer may include therein a heat transfer member formed of a material with high thermal conductivity.

In addition, the mask may further include a metal heat dissipation layer which is formed of a metal material with high thermal conductivity and is disposed between the phase-change material heat dissipation layer and the thermoelectric module so that one surface comes in contact with the thermoelectric module and the other surface comes in contact with the phase-change material heat dissipation layer.

In addition, the surface of the metal heat dissipation layer that comes in contact with the phase-change material heat dissipation layer may be formed of a concavo-convex structure.

In addition, the mask may further include a housing for positioning the phase-change material heat dissipation layer, and the phase-change material heat dissipation layer may be mountable on or separable from the housing.

In addition, the mask may further include an outer cover disposed at an upper portion of the phase-change material heat dissipation layer, and, in the outer cover, a fan configured to introduce outside air, exchange heat, and discharge heat generated at the second major surface to the outside may be installed.

In addition, the controller may control the thermoelectric module to cool the first major surface within a temperature range of −15° C. to 15° C.

In addition, the controller may control the thermoelectric module to maintain the cooling of the first major surface between 5 seconds, which is necessary for suppressing the pigmentation, and less than 300 seconds, at which damage to the skin begins.

In addition, another aspect of the present specification may provide a device for whitening skin, the device comprising, a contact layer having a lower portion being contacted with a face surface of a user, and provided as a flexible material for a tight-contact between the lower portion and the face surface, at least one thermoelectric module having two major surfaces including a first major surface disposed in contact with an upper portion of the contact layer and a second major surface positioned opposite to the first major surface, wherein the thermoelectric module cools the first major surface and applies, via the contact layer, a negative heat to a skin of the user, a phase-change material heat dissipation layer which is disposed at an upper portion of the thermoelectric module so as to come in contact with the second major surface of the thermoelectric module and includes a phase-change material which receives, via the second major surface, heat generated upon cooling of the first major surface and absorbs the received heat and maintains the temperature of the second major surface constant using latent heat at a melting point in order to maintain a difference between a temperature of the first major surface and a temperature of the second major surface to be less than a predetermined temperature, and a controller configured to control the thermoelectric module to cool the first major surface at a temperature at which pigmentation by melanocytes is suppressed in order to whiten the skin.

Hereinafter, a mask for whitening skin 10 according to an embodiment of the present specification will be described.

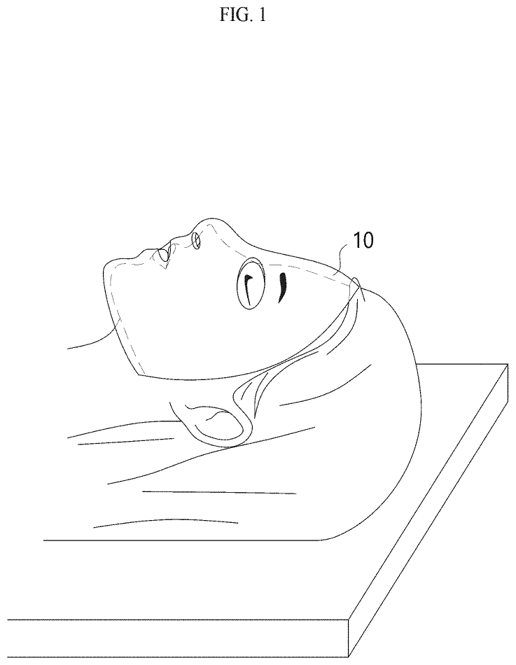

FIG. 1 is a view illustrating a state in which a user uses a mask for whitening skin according to an embodiment of the present specification.

Referring to FIG. 1, the mask for whitening skin 10 may have various effects on the skin of a user by cooling the skin of the user through a thermoelectric module (or a thermoelectric cooler) 30 positioned therein.

For example, by cooling the skin of the user at a temperature for suppressing pigmentation by melanocytes during a predetermined interval through the thermoelectric module 30 positioned therein, the mask for whitening skin 10 may reduce the amount of melanin produced from the melanocytes, reduce the amount of melanin transferred onto the skin, and whiten the skin of the user.

In addition, by cooling the skin of the user at a temperature at which it is possible to obtain additional effects during a predetermined interval through the thermoelectric module 30 positioned therein, the mask for whitening skin 10 may have additional effects, such as reduction of swelling and lipolysis, on the skin of the user.

In addition, by cooling the skin of the user within an activation temperature range of a functional material 21 during a predetermined interval through the thermoelectric module 30 positioned therein, the mask for whitening skin 10 may increase the activity of the functional material 21 that improves the facial skin of the user.

In addition, the mask for whitening skin 10 may have a shape corresponding to a facial region in order to be adhered to the facial region and evenly cool the skin of the user and may be provided in a form in which holes are formed around the eyes and lips because the eye and lip areas of the user are vulnerable to low temperature. For reasons such as improving adhesiveness, for convenience, the mask for whitening skin 10 may be provided in a form in which a hole is also formed around the nose area.

In addition, when a weight of the mask for whitening skin 10 is heavy due to a heat dissipation layer 40, the mask for whitening skin 10 may be used while the user is lying down. However, the present specification is not limited thereto.

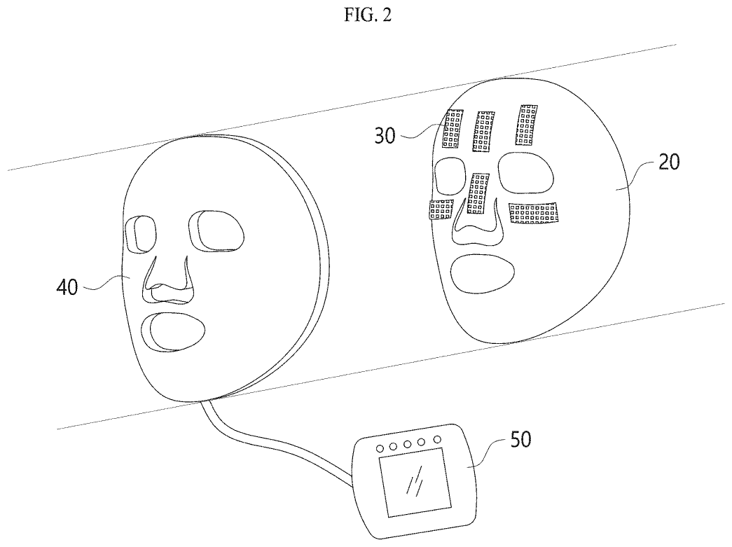

FIG. 2 is an exploded perspective view of the mask for whitening skin according to an embodiment of the present specification.

Referring to FIG. 2, the mask for whitening skin 10 may include a contact layer 20, at least one thermoelectric module 30, a heat dissipation layer 40, and a controller 50.

The contact layer 20 may have a lower portion coming in contact with a face of a user and be provided with a flexible material so as to be tightly contacted to the face. Also, the contact layer 20 may be provided with a material with high thermal conductivity in order to increase efficiency of cooling the skin of the user through the thermoelectric module 30. For example, the contact layer 20 may be provided with a material such as cotton, bio cellulose, and hydrogel.

In addition, the contact layer 20 may be provided with a small thickness so that negative heat is easily applied from the thermoelectric module 30 to the skin of the user. For example, the contact layer 20 may be provided with a thickness within 1 cm.

In addition, the contact layer 20 may be provided in a shape corresponding to the face of the user. Because the eye and lip areas of the user are vulnerable to low temperature, the contact layer 20 may be provided in a form in which holes are formed around the eyes and lips. For reasons such as improving adhesiveness, for convenience, the contact layer 20 may be provided in a form in which a hole is also formed around the nose area.

In addition, in order to improve the adhesiveness with the skin, the contact layer 20 may be provided together with a liquid that has viscosity. For example, the contact layer 20 may be provided in a state in which it is soaked with emulsion that has viscosity.

In addition, in order to obtain additional skin improvement effects, various functional materials 21 may be accommodated in the contact layer 20. For example, the contact layer 20 may be coated with a coating liquid that contains the functional material 21.

The functional material 21 is a material that has an effect of improving skin, and, for example, the functional material 21 may include an ingredient that helps protect against UV rays, an ingredient that prevents oxidation, an ingredient that conditions the skin, an ingredient that inhibits the action of bacteria, an ingredient that whitens the skin (arbutin, niacinamide, ascorbyl glucoside, or the like), an ingredient that reduces the size of pores, an ingredient that reduces wrinkles (retinol, adenosine, or the like), an ingredient that revitalizes the skin, an ingredient that prevents burning sensation, an ingredient that relieves pain, and the like. In addition, the functional material 21 may be formed of at least one of an ingredient that helps protect against UV rays, an ingredient that prevents oxidation, an ingredient that conditions the skin, an ingredient that inhibits the action of bacteria, an ingredient that whitens the skin, an ingredient that reduces the size of pores, an ingredient that reduces wrinkles, an ingredient that revitalizes the skin, an ingredient that prevents burning sensation, and an ingredient that relieves pain or a mixture of two or more thereof.

In addition, the contact layer 20 may be provided in various forms. The contact layer 20 may be provided so as to be fixed at the mask for whitening skin 10. Alternatively, the contact layer 20 may be provided so as to be temporarily disposed between the thermoelectric module 30 and the skin of the user when the mask for whitening skin 10 is worn. That is, the contact layer 20 may be independently configured with a structure that is attachable to or detachable from the mask 10 and may be separable from the mask 10.

For example, the contact layer 20 may be provided as an independent sheet that is disposed between the thermoelectric module 30 and the skin of the user when the mask for whitening skin 10 is worn. As another example, the contact layer 20 may be provided in the form of independent gel that is coated between the thermoelectric module 30 and the skin of the user when the mask for whitening skin 10 is worn.

When the contact layer 20 of the mask 10 is arbitrarily replaced by the user with another contact layer 20 which accommodates a functional material 21 that has side effects at low temperatures, irritation, damage, and the like may occur to the skin of the user as the skin of the user is cooled by the mask 10.

Therefore, according to an embodiment of the present specification, an identification tag 22 may be disposed at the contact layer 20 as a means for identifying the contact layer 20.

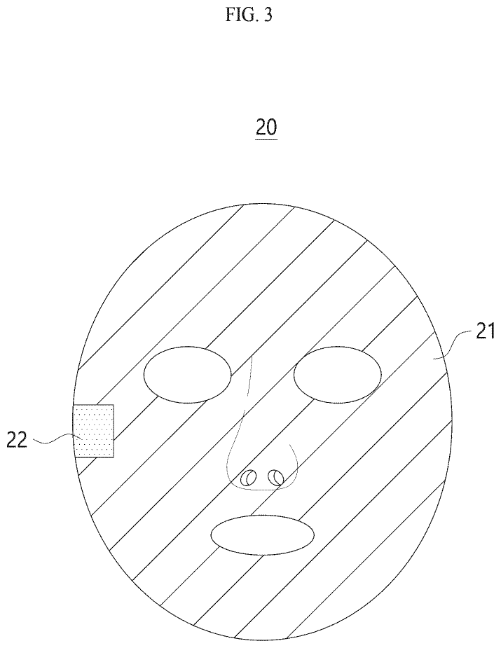

FIG. 3 is a view illustrating a contact layer at which an identification tag is disposed according to an embodiment of the present specification.

Referring to FIG. 3, the identification tag 22 may be disposed at the contact layer 20 as a means for identifying the contact layer 20.

The identification tag 22 may be disposed at any position on the contact layer 20 or may be disposed at a position corresponding to a site where a tag recognition module 74 configured to recognize the identification tag 22 is installed.

For example, the identification tag 22 may be provided as an electrically erasable programmable read-only memory (EEPROM), a radio-frequency identification (RFID) tag, or the like.

The identification tag 22 may have data relating to the contact layer 20 on which the identification tag 22 is disposed.

The identification tag 22 may have identification data relating to the contact layer 20 on which the identification tag 22 is disposed. For example, the controller 50 may control the thermoelectric module 30 so that power is applied to the thermoelectric module 30 only when the identification tag 22 is recognized by the tag recognition module 74 and the contact layer 20 is identifiable. That is, when the identification tag 22 is not recognized by the tag recognition module 74, the controller 50 may control so that power is not applied to the thermoelectric module 30.

The identification tag 22 may have identification data relating to the functional material 21 accommodated in the contact layer 20 on which the identification tag 22 is disposed. For example, when the identification tag 22 is recognized by the tag recognition module 74, the controller 50 may identify the functional material 21 accommodated in the contact layer 20 through the identification data that the identification tag 22 has.

The identification tag 22 may have data relating to a cooling condition of the thermoelectric module 30 (a cooling condition of a first major surface 31) that corresponds to the identification data relating to the functional material 21 accommodated in the contact layer 20 on which the identification tag 22 is disposed. For example, when the identification tag 22 is recognized by the tag recognition module 74, the controller 50 may obtain the data relating to the cooling condition of the thermoelectric module 30 (the cooling condition of the first major surface 31) that corresponds to the identification data that the identification tag 22 has and control the thermoelectric module 30 according to the obtained cooling condition.

The at least one thermoelectric module 30 may be formed to generate heat or absorb heat as power is applied thereto. For example, as power is applied thereto, the at least one thermoelectric module 30 may apply negative heat to the skin of the user via the contact layer 20.

A thermoelectric module may refer to a module that performs a thermoelectric operation, such as a power generating operation using a temperature difference or a heating/cooling operation using electrical energy, by using thermoelectric effects such as the Seebeck effect and the Peltier effect. Generally, most thermoelectric modules are provided in the form in which thermoelectric elements formed of n-type and p-type semiconductors are electrically connected on a flat substrate formed of ceramic material. However, in the present specification, the thermoelectric module 30 may include a thermoelectric module having flexibility. A thermoelectric element may be an element that causes the thermoelectric effects such as the Seebeck effect and the Peltier effect. Fundamentally, the thermoelectric element may include dissimilar materials constituting a thermoelectric couple that causes the thermoelectric effects. The thermoelectric couple may generate a temperature difference when electrical energy is applied thereto and, conversely, produce electrical energy when a temperature difference is applied thereto. Examples of the thermoelectric element may include a bismuth-antimony couple. Also, a pair of n-type semiconductor and p-type semiconductor may be mainly used as the thermoelectric element.

The at least one thermoelectric module 30 may have two major surfaces including the first major surface 31 disposed in contact with an upper portion of the contact layer 20 and a second major surface 32 positioned opposite to the first major surface 31. For example, the at least one thermoelectric module 30 may have two major surfaces 31 and 32 including the first major surface 31 disposed in contact with the upper portion of the contact layer 20 and the second major surface 32 positioned opposite to the first major surface 31 and, as power is applied, cool the first major surface 31 and apply negative heat to the skin of the user via the contact layer 20.

The pair of major surfaces 31 and 32 may include the first major surface 31 and the second major surface 32 which are disposed to be spaced apart to face each other. The first major surface 31 and the second major surface 32 may support a thermoelectric element or an electrode disposed therebetween. Also, the first major surface 31 and the second major surface 32 may perform a function of protecting a thermoelectric element or an electrode therein from the outside.

Also, the pair of major surfaces 31 and 32 may be provided with a material that facilitates heat conduction. For example, the pair of major surfaces 31 and 32 may be substrates formed of a copper material.

In addition, the at least one thermoelectric module 30 according to an embodiment of the present specification may be provided as a flexible thermoelectric module 30 that has flexibility. In order to be adhered well to the face of the user, the mask 10 may be manufactured in a complex shape that is curved or the like (e.g., a shape corresponding to the face). When the flexible thermoelectric module 30 is used, it is possible to reduce difficulties in manufacturing and provide the mask 10 having a shape that allows it to be adhered better to the face.

To this end, the pair of major surfaces 31 and 32 and the inside thereof of the thermoelectric module 30 may be provided with a material having flexibility. For example, the pair of major surfaces 31 and 32 may be polyimide (PI) films. Although thermal conductivity thereof is not that high, the PI film may be advantageous for heat conduction because it has high flexibility and may be manufactured with a small thickness.

Because a plurality of areas constituting a human body part have different skin characteristics, it is necessary to cool the respective areas of the body part differently. For example, conditions under which skin damage or suppression of pigmentation occurs may be different in a plurality of areas constituting the facial region. Therefore, according to an embodiment of the present specification, the plurality of thermoelectric modules 30 may be classified into a plurality of thermoelectric module groups 33, and the plurality of thermoelectric module groups 33 may be controlled by the controller 50 separately of other thermoelectric module groups 33. The thermoelectric module groups 33 may be formed of at least one thermoelectric module 30. Here, the plurality of thermoelectric module groups 33 may be divided corresponding to areas of the face of the user.

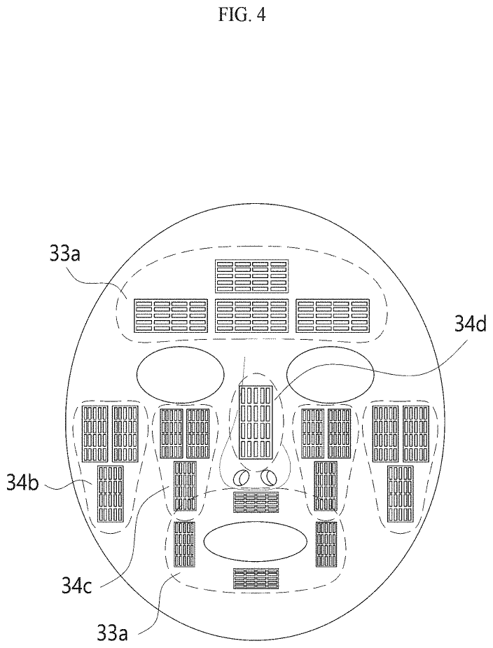

FIG. 4 is a view illustrating thermoelectric modules disposed inside a mask according to an embodiment of the present specification.

Referring to FIG. 4, a plurality of thermoelectric modules 30 may be classified into a plurality of thermoelectric module groups 33a, 33b, 33c, and 33d, corresponding to areas of the face of the user.

The thermoelectric module groups 33a, 33b, 33c, and 33d may be divided according to areas of the face of the user in which the thermoelectric module groups 33a, 33b, 33c, and 33d are disposed. For example, the thermoelectric module groups 33a, 33b, 33c, and 33d may be divided corresponding to areas with similar temperatures among areas constituting the face of the user.

Unlike the internal temperature of the human body which is in a range of 36° C. to 37° C., temperatures in a range of about 32° C. to 34° C. are distributed on the human skin. The temperature distribution on each area constituting the facial region of a person may vary among individuals. However, generally, the temperature distribution on the facial region is vertically symmetrical according to the shape in which blood vessels are distributed, and the facial region may be divided into a few regions with similar temperatures according to whether the temperature is high or low. The forehead area has a high temperature because there are many blood vessels and heat is unable to escape due to hair, and the jaw area has a high temperature because many blood vessels are distributed therearound. The areas at both sides of the nose have a relatively high temperature because relatively many blood vessels are distributed, and both cheek areas have a relatively low temperature because there are not many blood vessels distributed. The nose area has a lower temperature than other facial areas due to breathing in outside air. That is, generally, in the facial region of a person, the skin temperature is measured to be higher in the order of the forehead area, jaw area, areas at both sides of the nose, both cheek areas, and nose area. The facial region may be divided into the forehead area, jaw area, areas at both sides of the nose, both cheek areas, and nose area according to whether the temperature is high or low.

Therefore, according to an embodiment of the present specification, the plurality of thermoelectric modules 30 may be divided into a first thermoelectric module group 33a disposed in the forehead area and jaw area, a second thermoelectric module group 33b disposed in the areas at both sides of the nose, a third thermoelectric module group 33c disposed in both cheek areas, and a fourth thermoelectric module group 33d disposed in the nose area.

The plurality of thermoelectric module groups 33a, 33b, 33c, and 33d may be controlled by the controller 50 separately of other thermoelectric module groups 33a, 33b, 33c, and 33d. For example, the controller 50 may control the plurality of thermoelectric modules 30 such that the on/off, cooling interval, cooling temperature, operation mode, and the like of the plurality of thermoelectric module groups 33a, 33b, 33c, and 33d are controlled independently of those of other thermoelectric module group 33a, 33b, 33c, and 33d.

Regarding the plurality of thermoelectric module groups 33a, 33b, 33c, and 33d, a temperature at which the thermoelectric module group 33 is cooled may be lower in the thermoelectric module group 33 disposed in an area with a higher temperature in the facial region. That is, the controller 50 may control the plurality of thermoelectric module groups 33a, 33b, 33c, and 33d such that the temperature at which the first major surface 31 is cooled is lower in the thermoelectric module group 33 disposed in an area with a higher temperature in the facial region.

For example, the first major surface 31 of the first thermoelectric module group 33a disposed in the forehead and jaw areas which have high temperatures may be cooled at −15° C., and the first major surface 31 of the fourth thermoelectric module group 33d disposed in the nose area which has a low temperature may be cooled at 0° C.

Also, regarding the plurality of thermoelectric module groups 33a, 33b, 33c, and 33d, a cooling interval may be longer in the thermoelectric module group 33 disposed in an area with a higher temperature in the facial region. That is, the controller 50 may control the plurality of thermoelectric module groups 33a, 33b, 33c, and 33d such that the first major surface 31 is cooled for a longer interval in the thermoelectric module group 33 disposed in an area with a higher temperature in the facial region.

For example, the first thermoelectric module group 33a disposed in the forehead and jaw areas which have high temperatures may be cooled for a cooling interval of 120 seconds, and the fourth thermoelectric module group 33d disposed in the nose area which has a low temperature may be cooled for a cooling interval of 60 seconds.

However, the above-described thermoelectric module groups 33 are not limited thereto. There may be more or less thermoelectric module groups 33 than the above-described thermoelectric module groups 33, and each thermoelectric module group 33 is not limited by the above description.

Also, according to an embodiment of the present specification, one or more thermoelectric modules 30 may be disposed inside the mask 10 such that a separation distance therebetween is adjusted.

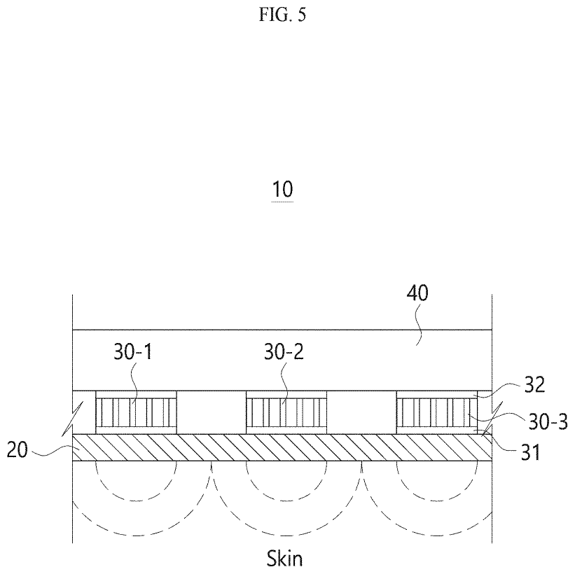

FIG. 5 illustrates a state in which negative heat of the mask is transferred to the skin according to an embodiment of the present specification.

The negative heat of the first major surface 31 of the thermoelectric module 30 is applied to the skin of the user via the contact layer 20. When the negative heat applied to the skin is transferred within the skin, the negative heat is transferred in a spherical shape.

Referring to FIG. 5, it can be seen that the negative heat is transferred in a semi-spherical shape to the skin from the thermoelectric module 30. That is, the skin temperature at a site at which the thermoelectric module 30 is disposed becomes different from the skin temperature at a site at which the thermoelectric module 30 is not disposed.

In a case in which a plurality of thermoelectric modules 30-1, 30-2, and 30-3 are disposed in the mask 10, when a separation distance between the thermoelectric modules 30-1, 30-2, and 30-3 is too long, the negative heat may not be properly transferred to some portions between the thermoelectric modules 30-1, 30-2, and 30-3, and whitening may occur partially, causing uneven skin tone. In order to prevent whitening from occurring partially, in the case in which the plurality of thermoelectric modules 30-1, 30-2, and 30-3 are disposed, the separation distance between the thermoelectric modules 30-1, 30-2, and 30-3 may be adjusted.

The plurality of thermoelectric modules 30-1, 30-2, and 30-3 may be disposed to be spaced apart at a separation distance that allows the negative heat to be applied to sites in between the thermoelectric modules 30-1, 30-2, and 30-3. For example, the plurality of thermoelectric modules 30-1, 30-2, and 30-3 may be disposed such that a separation distance between the thermoelectric modules 30-1, 30-2, and 30-3 is in a range of 1 mm to 50 mm.

The plurality of thermoelectric modules 30-1, 30-2, and 30-3 may be disposed on the basis of the skin temperatures at the sites in between the thermoelectric modules 30-1, 30-2, and 30-3 upon cooling and the skin temperatures at the sites at which the thermoelectric modules 30-1, 30-2, and 30-3 are disposed upon cooling.

The plurality of thermoelectric modules 30-1, 30-2, and 30-3 may be disposed to be spaced apart at a separation distance that allows a difference between the skin temperatures at the sites in between the thermoelectric modules 30-1, 30-2, and 30-3 upon cooling and the skin temperatures at the sites at which the thermoelectric modules 30-1, 30-2, and 30-3 are disposed upon cooling to be a predetermined temperature or less. For example, the first thermoelectric module 30-1 and the second thermoelectric module 30-2 may be disposed to be spaced apart at a separation distance that allows a difference between the skin temperature at a site in between the first thermoelectric module 30-1 and the second thermoelectric module 30-2 and the skin temperature at any one of a site at which the first thermoelectric module 30-1 is disposed and a site at which the second thermoelectric module 30-2 is disposed to be 10° C. or less.

As is well-known, when heat absorption occurs on one side of a thermoelectric element, heat generation occurs on the other side, and vice versa. For example, when heat absorption occurs on the first major surface 31 of the thermoelectric module 30 in order to cool the skin of the user, heat generation occurs on the second major surface 32 side of the thermoelectric module 30. When dissipation of the heat generated on the second major surface 32 side is not performed properly, the amount of power applied to the mask 10 may increase, and the cooling efficiency at the first major surface 31 may decrease. Therefore, the mask 10 may include the heat dissipation layer 40 configured to receive heat generated upon cooling of the first major surface 31 and dissipate the received heat.

The heat dissipation layer 40 may be disposed at an upper portion of the thermoelectric module 30 so as to come in contact with the second major surface 32 of the thermoelectric module 30 and may receive, via the second major surface 32, the heat generated upon the cooling of the first major surface 31 and dissipate the received heat. For example, the heat dissipation layer 40 may be formed of a material with high specific heat or thermal conductivity, or a fluid circulator, a fan, or the like that exchanges heat through a circulating fluid may be installed in the heat dissipation layer 40. Various heat dissipation means may be configured or installed in combination in the heat dissipation layer 40.

Also, in order to increase an area coming in contact with the outside so that heat dissipation is enhanced, the heat dissipation layer 40 may be provided in the shape protruding in a concavo-convex structure in which sides of the heat dissipation layer 40 coming in contact with the outside alternately protrude and indent.

Also, the heat dissipation layer 40 may be formed of a structure that is configured independently and is replaceable or detachable.

Generally, a temperature difference between a heat absorbing surface and a heat generating surface of a thermoelectric module has a characteristic of converging within a range of about 30° C. to less than 55° C. as power is applied. That is, when power is continuously applied to a thermoelectric module, a temperature difference between a heat absorbing surface and a heat generating surface of the thermoelectric module is maintained within the range of about 30° C. to less than 55° C. Therefore, when the temperature of the heat generating surface of the thermoelectric module is excessively high, the temperature of the heat absorbing surface of the thermoelectric module also increases, and a problem may occur in which the mask 10 is unable to cool the skin of the user at a target cooling temperature. In order to maintain the temperature difference between the heat absorbing surface and the heat generating surface within the range of about 30° C. to less than 55° C. and allow the thermoelectric module to be continuously used for cooling the skin of the user, the mask 10 may include the heat dissipation layer 40 configured to maintain the temperature difference between the first major surface 31 and the second major surface 32 to be a predetermined temperature or less (e.g., within the range of about 30° C. to less than 55° C.).

Therefore, according to an embodiment of the present specification, the heat dissipation layer 40 may be provided as a phase-change material heat dissipation layer 40a which is disposed at an upper portion of the thermoelectric module 30 so as to come in contact with the second major surface 32 of the thermoelectric module 30 and includes a phase-change material (PCM) which receives, via the second major surface 32, heat generated upon cooling of the first major surface 31 and absorbs the received heat and maintains the temperature of the second major surface 32 constant using latent heat at a melting point in order to maintain a difference between a temperature of the first major surface 31 and a temperature of the second major surface 32 to be less than a predetermined temperature.

A phase-change material is a material that has a large amount of latent heat, thus being capable of storing and releasing a large amount of energy upon a phase change. The phase-change material is a material that accumulates or releases heat through a process of changing from one phase to another, e.g., from solid to liquid, from liquid to solid, and the like. When the outside temperature drops below a melting point of the phase-change material, the phase-change material releases a large amount of latent heat as a phase change occurs. When the outside temperature rises to above the melting point of the phase-change material, the phase-change material absorbs a large amount of latent heat as a phase change occurs. Therefore, because the phase-change material absorbs or releases a large amount of heat as a phase change of the material occurs, the phase-change material is able to maintain a temperature corresponding to the melting point constant for a long period as compared with other materials. By using the phase-change material, the phase-change material heat dissipation layer 40a may maintain the temperature of the second major surface 32 constant and maintain the difference between the temperature of the first major surface 31 and the temperature of the second major surface 32 to be less than a predetermined temperature. Here, an appropriate value within the range of 30° C. to less than 55° C. may be selected as the predetermined temperature.

The phase-change material heat dissipation layer 40a may include a phase-change material that has a melting point within a specific range in order to maintain the difference between the temperature of the first major surface 31 and the temperature of the second major surface 32 to be less than a predetermined temperature. For example, the phase-change material heat dissipation layer 40a may include a phase-change material that has a melting point in a range of −15° C. to 40° C., such as C₁₂H₂₆, C₁₆H₃₄, and C₂₀H₄₂.

Also, the phase-change material heat dissipation layer 40a may include a phase-change material that has a mass determined on the basis of input power and use time of the thermoelectric module 30, an amount of heat absorbed by the first major surface 31, and the latent heat at the melting point of the phase-change material.

For example, a mass G of a phase-change material included in the phase-change material heat dissipation layer 40a may be determined by Equation 1 below.

$$G = \frac{( {Q}_{c} + P ) \cdot t}{( Δ H )} \qquad [ Equation 1 ]$$

Here, Q_{C} represents the amount of heat absorbed by the first major surface 31, P represents the input power of the thermoelectric module 30, t represents the use time of the thermoelectric module 30, and ΔH represents the latent heat at the melting point of the phase-change material.

The amount of heat Q_{C} absorbed by the first major surface 31 may be defined as a size of the quantity of heat absorbed per unit time by the first major surface 31 of the thermoelectric module 30.

For example, when the quantity of heat Q_{C} absorbed by the first major surface 31 is 3000 J/sec, the input power P of the thermoelectric module 30 is 2.3 W, the use time t of the thermoelectric module 30 is 40 seconds, and the latent heat ΔH at the melting point of the phase-change material is 230 J/g, according to Equation 1 above, the mass G of the phase-change material included in the phase-change material heat dissipation layer 40a is obtained as follows: G=(3000+2.3)×40/230=522.14 g.

Note that a method of determining the mass of the phase-change material included in the phase-change material heat dissipation layer 40a is not limited to the above-described method, and the mass may be determined using various other methods.

Also, the phase-change material heat dissipation layer 40a may be formed of a structure that is configured independently and is replaceable or detachable. For example, the phase-change material heat dissipation layer 40a may be configured in a block shape and connected to or separated from the mask 10.

Also, the phase-change material heat dissipation layer 40a may be disposed on an upper portion of the at least one thermoelectric module 30 and disposed in a housing (not illustrated), which is provided to position the phase-change material heat dissipation layer 40a, so that the phase-change material heat dissipation layer 40a is detachable or replaceable. In this way, the phase-change material heat dissipation layer 40a may be separated from the mask 10, cooled separately, and then re-mounted on the mask 10.

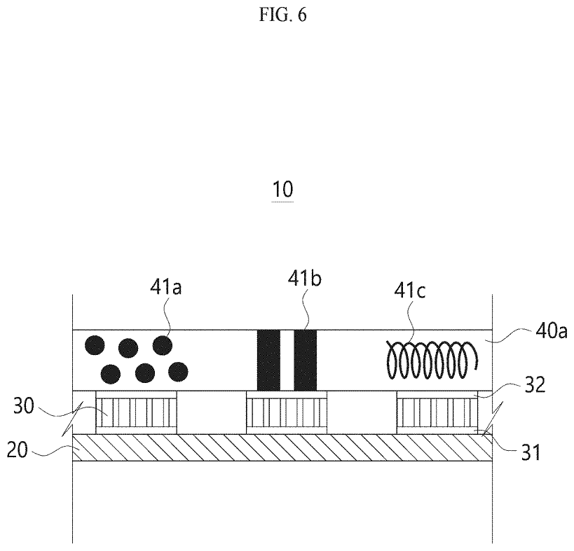

FIG. 6 illustrates a cross-sectional view of the mask 10 including a phase-change material heat dissipation layer that has a heat transfer member according to an embodiment of the present specification.

Referring to FIG. 6, the phase-change material heat dissipation layer 40a may include a heat transfer member 41 formed of a material with high thermal conductivity so that a heat exchange is facilitated inside the phase-change material heat dissipation layer 40a.

The heat transfer member 41 may be formed of a material with high thermal conductivity, e.g., a metal material such as aluminum and copper.

Also, the heat transfer member 41 may be provided in various forms. For example, the heat transfer member 41 may be provided in the form of a bead 41a, the form of a bar 41b that passes through the heat dissipation layer 40, or the form of a coil 41c.

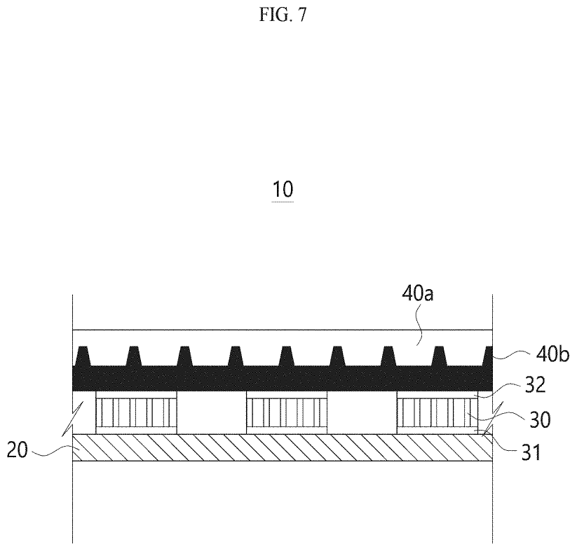

FIG. 7 illustrates a cross-sectional view of a mask including a metal heat dissipation layer according to an embodiment of the present specification.

Referring to FIG. 7, in order to improve a heat dissipation ability of the heat dissipation layer 40, a metal heat dissipation layer 40b may be disposed between the phase-change material heat dissipation layer 40a and the thermoelectric module 30 so that one surface comes in contact with the thermoelectric module 30 and the other surface comes in contact with the phase-change material heat dissipation layer 40a.

The metal heat dissipation layer 40b may be formed of a metal material with high thermal conductivity. For example, the metal heat dissipation layer 40b may be formed of a metal material such as aluminum and copper.

Also, the surface of the metal heat dissipation layer 40b that comes in contact with the phase-change material heat dissipation layer 40a may be formed of a concavo-convex structure. For example, the metal heat dissipation layer 40b may protrude in a concavo-convex structure in which sides of the metal heat dissipation layer 40b coming in contact with the phase-change material heat dissipation layer 40a alternately protrude and indent, and the metal heat dissipation layer 40b may protrude in various shapes.

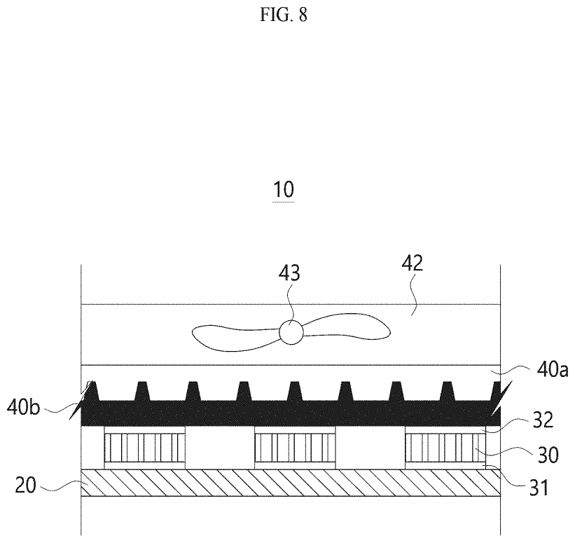

FIG. 8 illustrates a cross-sectional view of a mask including a fan according to an embodiment of the present specification.

Referring to FIG. 8, in order to improve the heat dissipation ability of the heat dissipation layer 40, an outer cover 42 may be disposed at an upper portion of the heat dissipation layer 40, and a fan 43 may be installed in the outer cover 42.

The outer cover 42 may be formed of a material with high thermal conductivity. For example, the outer cover 42 may be formed of a metal material such as aluminum and copper.

In order to enhance heat dissipation, the fan 43 may introduce outside air, exchange heat, and discharge heat generated at the second major surface 32 to the outside. The fan 43 may be installed not only in the outer cover 42 but also in other elements of the mask 10 such as the heat dissipation layer 40.

Also, a means for heat dissipation other than the fan 43 may be additionally provided in the outer cover 42. For example, a fluid circulator (not illustrated) configured to exchange heat through a fluid may be provided in the outer cover 42.

Also, a handle (not illustrated) that allows the mask 10 to be picked up may be provided in the outer cover 42. The handle may be provided with a material with low thermal conductivity in order to prevent the user from feeling uncomfortable due to hotness when the outer cover 42 becomes hot due to heat generated from the second major surface 32. For example, the handle may be provided with a material with low thermal conductivity such as rubber. Also, the handle may be provided to be disposed in other elements of the mask 10 such as the heat dissipation layer 40 instead of being disposed in the outer cover 42.

The controller 50 may be provided in a small size that is easy for the user to carry and may be connected to the mask 10 by wire or wirelessly to control the operation of the mask 10.

Also, the controller 50 may compare a measured temperature of the first major surface 31 of the thermoelectric module 30 with a predetermined temperature and measure a difference therebetween and may control the current or voltage supplied to the thermoelectric module 30 using various control methods including a proportional-integral-derivative (PID) control method in order to adjust a surface temperature of the first major surface 31 of the thermoelectric module 30.

For example, the controller 50 may control supply of power to at least one thermoelectric module 30 to adjust an operation mode in which a cooling temperature, a cooling interval, and the like of the first major surface 31 are set. Hereinafter, the operation mode refers to a mode in which the controller 50 controls the thermoelectric module 30 to cool the first major surface 31 according to predetermined temperature and interval.

The power supplied from the controller 50 to the thermoelectric module 30 or the like may be supplied by wire or wireless from the outside or supplied from a battery (not illustrated) separately accommodated in the mask 10.

Also, the controller 50 may be disposed inside the mask 10 or disposed at other places. Various modifications are possible.

Meanwhile, although not illustrated in the drawings, the mask for whitening skin 10 according to an embodiment of the present specification may further include additional elements.

The mask for whitening skin 10 may further include a skin temperature sensor 70 configured to measure a temperature of the skin of the user. The skin temperature sensor 70 may be disposed in any place, e.g., the contact layer 20, the thermoelectric module 30, the heat dissipation layer 40, the outer cover 42, or the like, which comes in contact with the skin of the user and at which it is possible to measure the temperature of the skin of the user. For example, the skin temperature sensor 70 may be disposed in the contact layer 20.

For example, the skin temperature sensor 70 may be a sensor such as a liquid expansion temperature sensor, a state change temperature sensor, a thermocouple, and a resistance temperature detector (RTD).

Also, the mask for whitening skin 10 may further include a second major surface temperature sensor 71 configured to measure a temperature of the second major surface 32 of the thermoelectric module 30. The second major surface temperature sensor 71 may be disposed in any place, e.g., the contact layer 20, the thermoelectric module 30, the heat dissipation layer 40, the outer cover 42, or the like, which comes in contact with the second major surface 32 and at which it is possible to measure the temperature of the second major surface 32. For example, the second major surface temperature sensor 71 may be disposed in the second major surface 32.

The second major surface temperature sensor 71 may be a sensor such as a liquid expansion temperature sensor, a state change temperature sensor, a thermocouple, and an RTD.

Also, the mask for whitening skin 10 may further include a vibration generating module (or a vibration generator) 72 configured to output vibration. The vibration generating module 72 may be disposed in any place, e.g., the contact layer 20, the thermoelectric module 30, the heat dissipation layer 40, the outer cover 42, or the like, at which it is possible to apply vibration to the face of the user. For example, the vibration generating module 72 may be installed to be attached to the thermoelectric module 30. For example, the vibration generating module 72 may be provided as an oscillator, a vibration motor, a haptic device, or the like.

The vibration generating module 72 may output vibration for notifying the user. For example, when the cooling of the first major surface 31 is completed (e.g., when the operation mode is ended), the vibration generating module 72 may output vibration for notifying that the cooling is ended.

Also, the vibration generating module 72 may output vibration for alleviating pain of the user while the skin of the user is being cooled. For example, the vibration generating module 72 may output vibration (e.g., a vibration massage) and apply the vibration to the skin of the user while the thermoelectric module 30 applies negative heat to the skin of the user.

The vibration generating module 72 may output stronger vibration as the intensity of negative heat that the thermoelectric module 30 applies to the skin of the user is higher (i.e., the cooling temperature of the first major surface 31 is lower or the cooling interval of the first major surface 31 is longer).

Also, the vibration generating module 72 may output the vibration for notifying the user and the vibration for alleviating the pain of the user in different manners.

Also, the mask for whitening skin 10 may further include a touch sensing module (or a touch sensor) 73 configured to detect contact of the skin of the user with the mask 10.

The touch sensing module 73 may be disposed in any place, the contact layer 20, the thermoelectric module 30, the heat dissipation layer 40, the outer cover 42, or the like, at which it is possible to come in contact with the skin of the user. For example, the touch sensing module 73 may be disposed in the contact layer 20.

The touch sensing module 73 may generate a skin detection signal when the skin of the user is detected. Alternatively, the touch sensing module 73 may generate a skin non-detection signal when the skin of the user is not detected.

The touch sensing module 73 may be used as a means for determining a point in time at which cooling of the first major surface 31 starts. For example, the touch sensing module 73 may generate a skin detection signal upon detecting the skin of the user and transmit the corresponding signal to the controller 50, and, when the skin detection signal is received, the controller 50 may control the thermoelectric module 30 to start the cooling of the first major surface 31.

For example, the touch sensing module 73 may be formed of at least one touch sensor configured to detect contact using electrical characteristics that occur upon contact with the skin of the user or formed of a heat sensor configured to detect human heat.

Also, the mask. for whitening skin 10 may further include the tag recognition module 74 configured to recognize the identification tag 22.

The tag recognition module 74 may be disposed in any place, i.e., the thermoelectric module 30, the heat dissipation layer 40, the controller 50, or the like, at which it is possible to recognize the identification tag 22. For example, the tag recognition module 74 may be installed in an inner space of the mask 10 that corresponds to a place where the identification tag 22 is positioned on the contact layer 20.

The tag recognition module 74 may recognize the identification tag 22. For example, the tag recognition module 74 may read data of the identification tag 22 disposed at the contact layer 20 and identify the identification tag 22.

The tag recognition module 74 may read or write data of the identification tag 22 disposed at the contact layer 20. For example, the tag recognition module 74 may recognize the identification tag 22 and read identification data that the identification tag 22 has, and the controller 50 may, from the identification data, identify the functional material 21 accommodated in the contact layer 20. As another example, the tag recognition module 74 may read data relating to a cooling condition of the thermoelectric module 30 (a cooling condition of a first major surface 31) that corresponds to the identification data that the identification tag 22 has, and the controller 50 may control the thermoelectric module 30 according to the cooling condition data.

The tag recognition module 74 may be provided as an RFID reader, an RFID reader writer, an EEPROM, or the like.

Also, although not illustrated in the drawings, the mask for whitening skin 10 according to an embodiment of the present specification may further include a strap, a band, or the like that provides tension so that, upon use of the mask for whitening skin 10 by the user, the mask for whitening skin 10 is adhered to the skin of the user.

Also, the mask for whitening skin 10 is not limited by the above-described configuration. The mask for whitening skin 10 may include elements more or less than those described above, and each element is not limited by the above description.

Hereinafter, the controller 50 will be described in detail.

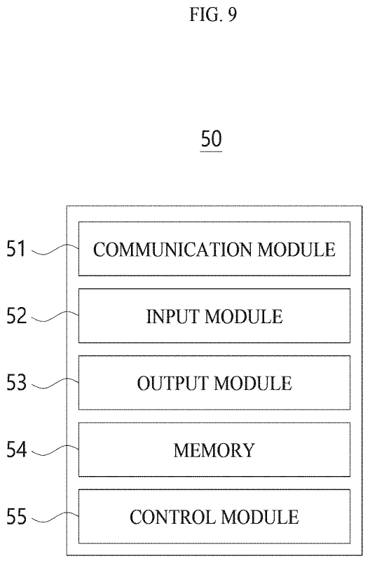

FIG. 9 is a block diagram of a controller according to an embodiment of the present specification.

Referring to FIG. 9, the controller 50 according to an embodiment of the present specification may include a communication module 51, an input module 52, an output module 53, a memory 54, and a control module 55.

The communication module 51 may perform communication with an external device. For example, the controller 50 may exchange predetermined data with an external device via the communication module 51.

Specifically, the communication module 51 may exchange data relating to the operation mode with an external device.

The communication module 51 may send data relating to the operation mode that is received from the input module 52 to an external device (e.g., a server) or may obtain data relating to the operation mode from an external device (e.g., a server).

For example, the communication module 51 may obtain, from an external device, data that provides an operation mode for improving the skin of each user on the basis of results of monitoring skin conditions of the user. As another example, the communication module 51 may obtain, from an external device, data relating to an operation mode that causes the temperature to be sequentially maintained at 0° C. for 30 seconds, 5° C. for 40 seconds, and 10° C. for 60 seconds.

The communication module 51 may communicate with an external device such as another local device and/or a server. The communication module 51 may include one or more modules that allow the communication. The communication module 51 may communicate with an external device by wire or wirelessly. To this end, the communication module 51 may be formed as a wired communication module that accesses the Internet through a local area network (LAN), a mobile communication module such as a long term evolution (LTE) communication module that transmits and receives data by connecting to a mobile communication network via a mobile communication base station, a short-range communication module using a wireless local area network (WLAN)-based communication method such as Wi-Fi or a wireless personal area network (WPAN)-based communication method such as Bluetooth or ZigBee, a satellite communication module using a global navigation satellite system (GNSS) such as a global positioning system (GPS), or a combination thereof.

The input module 52 may receive a user input from a user. The user input may be in various forms such as a key input, a touch input, and a voice input. For example, the input module 52 may receive, via a key input button, a user's input on a selection of image resolution.

Also, the input module 52 may receive, from a user, data relating to a user settings operation mode. Specifically, the input module 52 may receive data relating to a cooling interval, a cooling temperature, a cooling order, a cooling area, and the like of the user settings operation mode.

For example, the input module 52 may receive, from a user, data that provides an operation mode for improving the skin of each user on the basis of results of monitoring skin conditions of the user. As another example, the input module 52 may receive data relating to an operation mode that causes the temperature to be sequentially maintained at 0° C. for 30 seconds, 5° C. for 40 seconds, and 10° C. for 60 seconds.

Typical examples of the input module 52 not only include a keypad, a keyboard, and a mouse in conventional forms, but also include a touch sensor that detects a user's touch, a microphone that receives a voice signal, a camera that recognizes a gesture or the like through image recognition, a proximity sensor formed of an illuminance sensor, an infrared sensor, or the like that detects a user's approach, a motion sensor that recognizes a user's motion through an acceleration sensor, a gyro sensor, or the like, and various other input means that detect or receive various forms of user input. Here, the touch sensor may be implemented as a piezoelectric or capacitive touch sensor that senses a touch through a touch panel or a touch film attached to a display panel, an optical touch sensor that senses a touch by an optical method, or the like.

The output module 53 may include a display that outputs an image, a speaker that outputs sound, a haptic device that generates vibration, and various other output means.

The display is a concept that encompasses image display devices in a broad sense that include a liquid crystal display (LCD), a light emitting diode (LED) display, an organic light emitting diode (OLED) display, a flat panel display (FPD), a transparent display, a curved display, a flexible display, a 3D display, a holographic display, a projector, and various other devices capable of performing an image output function. The display may also be in the form of a touch display that is integrally formed with the touch sensor of the input module 52. In addition, instead of being implemented in the form of a device that outputs information to the outside by itself, the output module 53 may be implemented in the form of an output interface (a universal serial bus (USB) port, a Personal System (PS)/2 port, or the like) that connects an external output device to an image processing device.

The memory 54 may store information relating to operation of the controller 50.

Specifically, the memory 54 may store data relating to an operation mode obtained from an external device, Also, the memory 54 may store data relating to an operation mode input from the input module 52.

For example, the memory 54 may store data that provides an operation mode for improving the skin of each user on the basis of results of monitoring skin conditions of the user. As another example, the memory 54 may store data relating to an operation mode that causes the temperature to be sequentially maintained at 0° C. for 30 seconds, 5° C. for 40 seconds, and 10° C. for 60 seconds.

Also, the memory 54 may store an operating system (OS), firmware, middleware, and various programs that support the same for driving the controller 50 or store data or the like received from other external devices such as a user terminal.

The control module 55 may be involved in the overall operation of the elements of the controller 50. Therefore, unless otherwise stated, the operation of the controller 50 may be interpreted as being caused by the control module 55.

The control module 55 may be implemented as a computer or a similar device according to hardware, software, or a combination thereof. The hardware of the controller may be provided in the form of an electronic circuit such as a central processing unit (CPU), a micro control unit (MCU), and a chip that processes an electrical signal and performs a control function. The software of the controller may be provided in the form of a program that drives the hardware of the controller.

However, the controller 50 is not limited by the above-described configuration. The controller 50 may include elements more or less than those described above, and each element is not limited by the above description.

Hereinafter, a first embodiment in which the controller 50 controls the temperature of the first major surface 31 of the thermoelectric module 30 will be described.

### First Embodiment

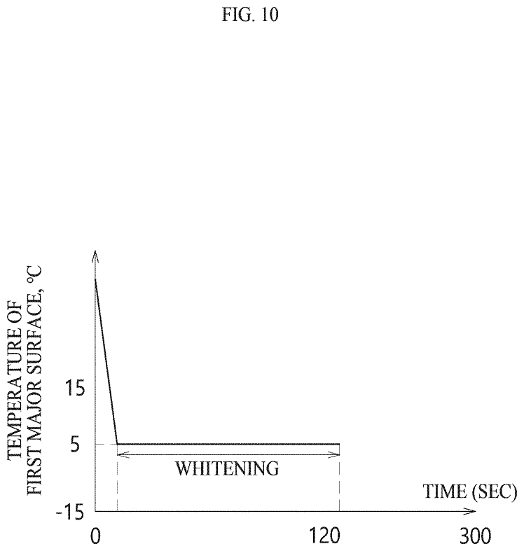

FIG. 10 is a graph showing temperature changes over time of a first major surface of a thermoelectric module according to a first embodiment of the present specification.

When the user's skin is cooled too much, skin damage may occur due to skin freezing or the like. When the user's skin is cooled too lightly, pigmentation by melanocytes may not be suppressed by the cooling.

Therefore, the controller 50 according to an embodiment of the present specification may control the thermoelectric module 30 in a whitening mode in which the first major surface 31 is cooled under cooling conditions for preventing skin damage while suppressing pigmentation by melanocytes. Referring to FIG. 10, it can be seen that the thermoelectric module 30 is controlled by the controller 50 and the whitening mode is performed in which the first major surface 31 is cooled for 120 seconds at a cooling temperature of 5° C.

Hereinafter, an interval during which the first major surface 31 is cooled in the whitening mode will be referred to as “whitening interval”, a temperature of the first major surface 31 while the whitening mode is performed will be referred to as “whitening temperature,” and a temperature range of the first major surface 31 while the whitening mode is performed will be referred to as “whitening temperature range.”

The controller 50 may control the thermoelectric module 30 to cool the first major surface 31 within a specific temperature range in order to prevent skin damage due to cooling while suppressing pigmentation by melanocytes on the user's skin. For example, the controller 50 may control the thermoelectric module 30 to cool the first major surface 31 at a cooling temperature such that the skin of the user reaches a target temperature at which pigmentation by melanocytes is suppressed, wherein the cooling temperature is in a range of −15° C. to 15° C., which is a whitening temperature range, and lower than the target temperature. This is because skin damage may occur due to freezing of skin tissues when the temperature of the first major surface 31 is too low, and the effect of suppressing pigmentation by melanocytes may be small when the temperature of the first major surface 31 is too high.

Here, the target temperature may be in a range of 4° C. to 27° C. Because the skin whitening effect by cooling of the skin increases as the temperature of the skin is maintained low, the target temperature may be a temperature in a range of 27° C. or lower in order for the skin whitening effect by the cooling of the skin to occur substantially. Also, although the actual freezing of human skin tissues does not occur before the skin temperature reaches a temperature in a range of −4° C. to −10° C., tissue damage may occur due to ischemia and blood clots in small blood vessels when the skin temperature drops below 4° C. Thus, the target temperature may be a temperature in a range of 4° C. or higher in order to prevent skin damage due to the cooling of the skin.

However, the cooling temperature of the first major surface 31 that is controlled by the controller 50 does not have to be limited by the above description and may vary according to circumstances. For example, the cooling temperature of the first major surface 31 may be maintained within a temperature range of −30° C. to 35° C. Also, the target temperature does not have to be limited by the above description and may vary according to circumstances. For example, the target temperature may be maintained within a temperature range of −10° C. to 35° C.

Also, in order to suppress pigmentation by melanocytes while preventing skin damage due to cooling, the controller 50 may control the thermoelectric module 30 to maintain a whitening interval, during which the cooling of the first major surface 31 occurs, to be longer than a first interval, which is necessary for suppressing the pigmentation, and shorter than a second interval, during which skin damage begins. For example, in order to prevent skin damage while suppressing pigmentation by melanocytes, the controller 50 may control the thermoelectric module 30 to maintain a cooling interval of the first major surface 31 between 5 seconds, which is necessary for suppressing the pigmentation, and less than 300 seconds, at which damage to the skin begins. As another example, the controller 50 may control the thermoelectric module 30 to maintain the cooling interval of the first major surface 31 between 5 seconds, which is necessary for suppressing the pigmentation, and less than 900 seconds, at which microscopic damage to the skin may begin.

Here, the first interval may indicate a minimum interval necessary for suppressing pigmentation by cooling melanocytes. When the skin is cooled for an interval shorter than the first interval, it is not possible to suppress the pigmentation because the melanocytes are not sufficiently cooled. For example, for the skin whitening effect to substantially occur by cooling the skin, the first interval may be at least 5 seconds. However, the first interval may vary according to circumstances. For example, the first interval may be at least 0.5 seconds, which is the minimum interval necessary for the skin whitening effect to occur by the cooling the skin.

Also, the second interval may indicate an interval during which skin damage begins due to the cooling of the skin. When the skin is cooled for an interval longer than the second interval, damage to the skin may occur. For example, the second interval may be 300 seconds at maximum in order to prevent tissue damage due to ischemia and blood clots in small blood vessels or the like that occur as the skin is cooled for a long period. However, the second interval may vary according to circumstances. For example, the second interval may be, at maximum, 900 seconds, which is an interval during which, not only direct damage to the skin, but also microscopic damage to the skin may begin due to cooling the skin for a long period.

The cooling interval of the first major surface 31 that is controlled by the controller 50 is not necessarily limited by the above description and may be determined in various other ways.

Also, the controller 50 may control the thermoelectric module 30 to cool the first major surface 31 for a longer cooling interval as the cooling temperature of the first major surface 31 is higher. For example, the controller 50 may control the thermoelectric module 30 to cool the first major surface 31 for a longer interval when the cooling temperature of the first major surface 31 is 5° C. as compared with when the cooling temperature of the first major surface 31 is −15° C.

Also, the controller 50 according to an embodiment of the present specification may control the thermoelectric module 30 on the basis of a skin temperature measured by the skin temperature sensor. To this end, the controller 50 may store, in the memory 54, result values according to input in the form of functions or a look-up table and use the results values upon calculation. For example, the memory 54 may store a current/voltage table relating to current and/or voltage for each skin temperature and intensity. The controller 50 may determine the size of current/voltage to be applied by referring to the current/voltage table on the basis of a measured skin temperature.

In order to suppress pigmentation by melanocytes while preventing damage to the skin due to cooling, the controller 50 may control the thermoelectric module 30 to change a cooling condition of the first major surface 31 on the basis of a skin temperature measured by the skin temperature sensor.

The controller 50 may control the thermoelectric module 30 to change a cooling temperature of the first major surface 31 on the basis of a skin temperature measured by the skin temperature sensor. For example, the controller 50 may control the thermoelectric module 30 to lower the cooling temperature of the first major surface 31 when the measured skin temperature is higher than the target temperature and raise the cooling temperature of the first major surface 31 when the measured skin temperature is lower than the target temperature. As another example, when the measured skin temperature has reached the target temperature, the controller 50 may control the thermoelectric module 30 to cool the first major surface 31 at a cooling temperature that allows the measured skin temperature to be maintained at the target temperature.

The controller 50 may control the thermoelectric module 30 to change a cooling interval of the first major surface 31 on the basis of a skin temperature measured by the skin temperature sensor. For example, the controller 50 may control the thermoelectric module 30 to extend the cooling interval of the first major surface 31 when the measured skin temperature is higher than the target temperature and shorten the cooling interval of the first major surface 31 when the measured skin temperature is lower than the target temperature.

Also, the controller 50 may control the thermoelectric module 30 so that an interval during which the measured skin temperature is maintained at the target temperature is longer than an interval necessary for suppressing the pigmentation and shorter than an interval during which damage to the skin begins. For example, the controller 50 may control the thermoelectric module 30 so that the measured temperature is maintained at the target temperature (in the range of 4° C. to 27° C.) for 4 seconds to 120 seconds.

However, the first embodiment is not necessarily limited to the above-described method and may be practiced using various other methods, such as that in which the first embodiment is combined with other elements, according to circumstances.

### Second Embodiment

Hereinafter, a second embodiment in which the controller 50 controls the temperature of the first major surface 31 of the thermoelectric module 30 will be described. Descriptions overlapping those of the previous embodiment will be omitted. That is, descriptions of the technical ideas relating to the previous embodiment which may apply identically to the second embodiment will be omitted.

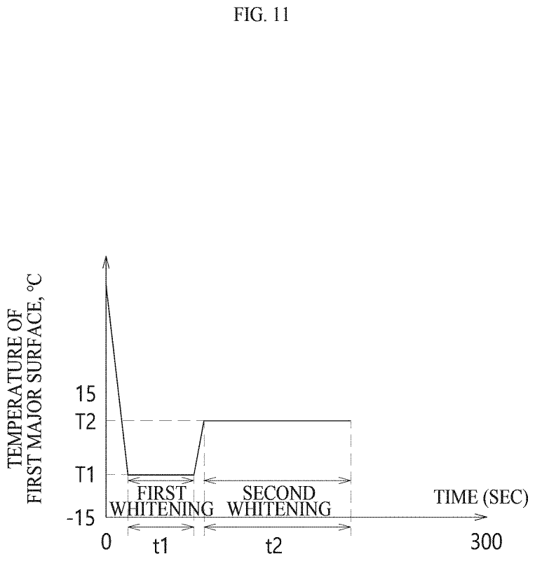

FIG. 11 is a graph showing temperature changes over time of a first major surface of a thermoelectric module according to a second embodiment of the present specification.

When the mask 10 cools the skin of a user using only one whitening mode, it is difficult for whitening of the skin to be performed efficiently. This is because the user's skin is prone to damage when the mask 10 cools the user's skin only using a whitening mode having a very low temperature, and the effect of suppressing pigmentation by melanocytes may decrease when the mask 10 cools the user's skin only using a whitening mode having a very high temperature.

Specifically, first, the mask 10 may cool the user's skin at a first whitening temperature, which is lower than a second whitening temperature, to rapidly decrease the temperature of the user's skin and increase the skin whitening effect. However, here, because the skin may be damaged due to low temperature when the mask 10 cools the user's skin at the first whitening temperature for a long period, a first whitening interval during which the first whitening temperature is maintained may be maintained relatively short.

Next, the mask 10 may cool the user's skin at the second whitening temperature which is higher than the first whitening temperature so that the skin whitening effect is increased by reducing damage to the skin due to low temperature and cooling the user's skin for a longer interval. That is, a second whitening interval during which the mask 10 maintains a second whitening mode may be longer than the first whitening interval during which the mask 10 maintains a first whitening mode.

Therefore, for efficient skin whitening, the controller 50 according to an embodiment of the present specification may control the thermoelectric module 30 to sequentially perform the first whitening mode and the second whitening mode which have different cooling conditions. Referring to FIG. 11, it can be seen that the thermoelectric module 30 is controlled by the controller 50 and sequentially performs the first whitening mode, in which the first major surface 31 is cooled for an interval t1 at a cooling temperature T1, and the second whitening mode, in which the first major surface 31 is cooled for an interval t2 longer than the interval t1 for at a cooling temperature T2 higher than the cooling temperature T1.

For efficient skin whitening, the controller 50 may control the thermoelectric module 30 to sequentially perform the first whitening mode and the second whitening mode which have different cooling conditions.

For example, first, the controller 50 may control the thermoelectric module 30 to perform the first whitening mode in which cooling is performed at a first whitening temperature which is lower than a second whitening temperature for a first whitening interval which is shorter than a second whitening interval.

Next, the controller 50 may control the thermoelectric module 30 to perform the second whitening mode in which cooling is performed at the second whitening temperature higher than the first whitening temperature for the second whitening interval longer than the first whitening interval.

Also, the controller 50 may control the thermoelectric module 30 using various other combinations of whitening modes. For example, the controller 50 may control the thermoelectric module 30 using a plurality of whitening modes more than two whitening modes or may sequentially repeat each whitening mode.

However, the second embodiment is not necessarily limited to the above-described method and may be practiced using various other methods, such as that in which the second embodiment is combined with other elements, according to circumstances.

### Third Embodiment

Hereinafter, a third embodiment in which the controller 50 controls the temperature of the first major surface 31 of the thermoelectric module 30 will be described. Descriptions overlapping those of the previous embodiments will be omitted. That is, descriptions of the technical ideas relating to the previous embodiments which may apply identically to the third embodiment will be omitted.

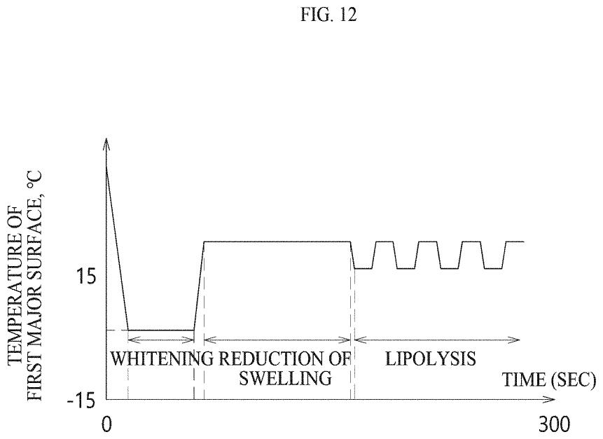

FIG. 12 is a graph showing temperature changes over time of a first major surface of a thermoelectric module according to a third embodiment of the present specification.

Generally, when the user's skin is cooled, additional effects such as reduction of swelling and lipolysis may occur in addition to the skin whitening effect. However, an optimum temperature at which the skin whitening effect occurs is slightly different from an optimum temperature at which additional effects such as reduction of welling and lipolysis occur.

Generally, a cooling temperature for causing the swelling reduction effect and lipolytic effect is higher than a cooling temperature for causing the whitening effect, and a cooling interval for causing the swelling reduction effect and lipolytic effect is longer than a cooling interval for causing the whitening effect. Here, when the user's skin is cooled by repeatedly raising and dropping the temperature, the lipolytic effect may be increased.

Therefore, the controller 50 according to an embodiment of the present specification may control the thermoelectric module 30 to cool the first major surface 31 also under conditions that cause other additional effects. For example, the controller 50 may control the thermoelectric module 30 to cool the first major surface 31 under conditions that cause effects such as reduction of swelling and lipolysis.

Referring to FIG. 12, it can be seen that the thermoelectric module 30 is controlled by the controller 50 and, after performing the whitening modes, sequentially performs a swelling reduction mode in which the first major surface 31 is cooled under a cooling condition that causes the swelling reduction effect and a lipolysis mode in which the first major surface 31 is cooled under a cooling condition that causes the lipolytic effect.

The controller 50 may control the thermoelectric module 30 to cool the first major surface 31 under a cooling condition for reducing swelling on the user's skin.

The controller 50 may control the thermoelectric module 30 to perform a swelling reduction mode in which the first major surface 31 is cooled within a temperature range that is higher than a whitening temperature range of the whitening modes. For example, the controller 50 may control the thermoelectric module 30 to cool the first major surface 31 within a temperature range of 10° C. to 20° C. that is higher than the whitening temperature range of the whitening modes.

The controller 50 may control the thermoelectric module 30 to perform a swelling reduction mode in which the first major surface 31 is cooled for a longer interval than the whitening intervals of the whitening modes. For example, the controller 50 may control the thermoelectric module 30 to cool the first major surface 31 for an interval in a range of 4 seconds to 20 minutes that is longer than the whitening intervals of the whitening modes.

The controller 50 may control the thermoelectric module 30 to cool the first major surface 31 under a cooling condition for lipolysis of the user's skin.

The controller 50 may control the thermoelectric module 30 to perform a lipolysis mode in which the first major surface 31 is cooled within a temperature range higher than the whitening temperature range of the whitening modes. For example, the controller 50 may control the thermoelectric module 30 to cool the first major surface 31 within a temperature range of 10° C. to 20° C. that is higher than the whitening temperature range of the whitening modes. Also, the controller 50 may control the thermoelectric module 30 to cool the first major surface by repeatedly raising and dropping the temperature within the temperature range of 10° C. to 20° C.

Also, the controller 50 may control the thermoelectric module 30 to perform a lipolysis mode in which the first major surface 31 is cooled for a longer interval than the whitening intervals of the whitening modes. For example, the controller 50 may control the thermoelectric module 30 to cool the first major surface 31 for an interval in a range of 4 seconds to 2 hours that is longer than the whitening intervals of the whitening modes.

Also, the controller 50 may control the thermoelectric module 30 to perform at least one operation mode of the whitening modes, the swelling reduction mode, and the lipolysis mode. For example, the controller 50 may control the thermoelectric module 30 to sequentially perform the swelling reduction mode and the lipolysis mode after performing the whitening modes.

Also, the controller 50 may control the thermoelectric module 30 to perform at least one of the swelling reduction mode and the lipolysis mode simultaneously with the whitening mode during at least a portion of the whitening interval during which the whitening mode is performed. That is, the controller 50 may control the thermoelectric module 30 so that the whitening temperature range overlaps with the temperature range of the swelling reduction mode and/or the lipolysis mode during at least a portion of the whitening interval during which the whitening mode is performed.

Also, the controller 50 may control the thermoelectric module 30 to perform the operation modes in various other combinations. For example, the controller 50 may control the thermoelectric module 30 to sequentially repeat the whitening modes, the swelling reduction mode, and the lipolysis mode.

Also, the controller 50 may control the thermoelectric module 30 to perform various operation modes for improving the skin, such as a skin regeneration mode and a skin elasticity mode, other than the whitening modes, the swelling reduction mode, and the lipolysis mode.

However, the third embodiment is not necessarily limited to the above-described method and may be practiced using various other methods, such as that in which the third embodiment is combined with other elements, according to circumstances.

### Fourth Embodiment

Hereinafter, a fourth embodiment in which the controller 50 controls the temperature of the first major surface 31 of the thermoelectric module 30 will be described. Descriptions overlapping those of the previous embodiments will be omitted. That is, descriptions of the technical ideas relating to the previous embodiments which may apply identically to the fourth embodiment will be omitted.

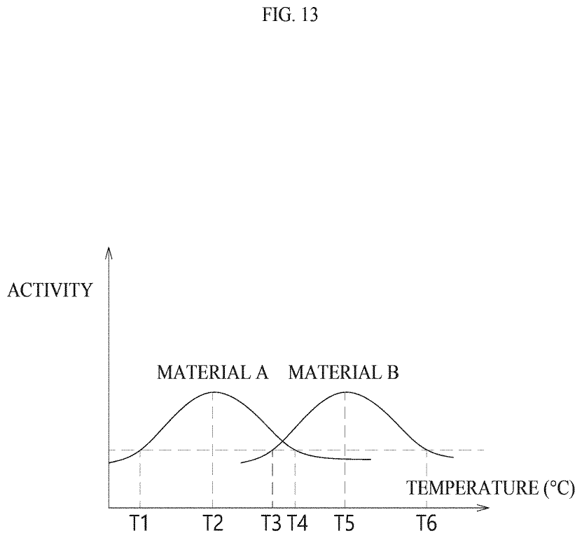

FIG. 13 is an example graph showing activity of a functional material according to temperature.

Referring to FIG. 13, the activity of a functional material may change according to temperature. A temperature range in which a functional material is active and a temperature at which the activity is maximum may be different for each functional material. That is, an activation temperature range, in which the activity is at a predetermined level or higher (e.g., nine-tenth of the maximum activity or higher), and a maximum activation temperature, at which the activity is maximum, may be different for each functional material. For example, from the graph of FIG. 13, it can be seen that functional material A has an activation temperature range ranging from T1 to T4 and maximum activity at T2, and functional material B has an activation temperature range ranging from T3 to T6 and maximum activity at T5.

A functional material 21 may be a material whose function is further activated at low temperature. For example, the functional material 21 may be a whitening functional material whose whitening effect is further enhanced (whitening function is further activated) at low temperature. As an example, which is not limiting, the functional material 21 may include resorcinol and similar derivatives (hexyl resorcinol, butyl resorcinol, phenylethyl resorcinol, resorcinol acetate, and other similar derivatives) and may be formed of one of the materials or a mixture of two or more thereof.

As another example, the functional material 21 may be a whitening functional material that further reduces irritation (e.g., irritation due to cooling) and whose effective concentration increases at low temperature. As an example, which is not limiting, the functional material 21 may include and be formed of one of, or a mixture of two or more of, resorcinol and similar derivatives (hexyl resorcinol, butyl resorcinol, phenylethyl resorcinol, resorcinol acetate, and other similar derivatives), niacinamide and a composition containing the same, magnesium ascorbylphosphate and a composition containing the same, ascorbyl glucoside and a composition containing the same, ascorbyl tetraisopalmitate/dipalmitate and a composition containing the same, arbutin and a composition containing the same, α-bisabolol and a composition containing the same, ethyl ascorbyl ether and a composition containing the same, polyphenol derivatives and a composition containing the same, L-glutathione and a composition containing the same, tranexamic acid and a composition containing the same, 4-methoxysalicylic acid potassium salt (KCl) derivatives and a composition containing the same, glycyrrhizine and a composition containing the same, azelaic acid, azelaic acid derivatives (e.g., azeloyl diglycine) and a composition containing the same, nicotinamide, nicotinamide derivatives and a composition containing the same, resveratrol, resveratrol derivatives and a composition containing the same, glycyrrhiza flavonoids, ellagic acid and a composition containing the same, papain and a composition containing the same, mandelic acid, mandelic acid derivatives and a composition containing the same, heptapeptide-1 and a composition containing the same, kojic acid, kojic acid derivatives and a composition containing the same, and plant extracts and a composition containing the same that contain all or some of the following ingredients: jasmine extract, mulberry extract, paper mulberry extract, licorice extract, ginseng extract, salvia miltiorrhiza extract, corn extract, chrysanthemum extract, bark root extract, thyme extract, white fresh root extract, polygon extract, magnolia tree extract, angelica root extract, phyllanthus emblica (fruit) extract, and citrus extract.

However, the graph of FIG. 13 is merely a schematic graph illustrated for description, and the fourth embodiment is not limited thereto. The activity of the actual functional material according to temperature may be different from the graph.

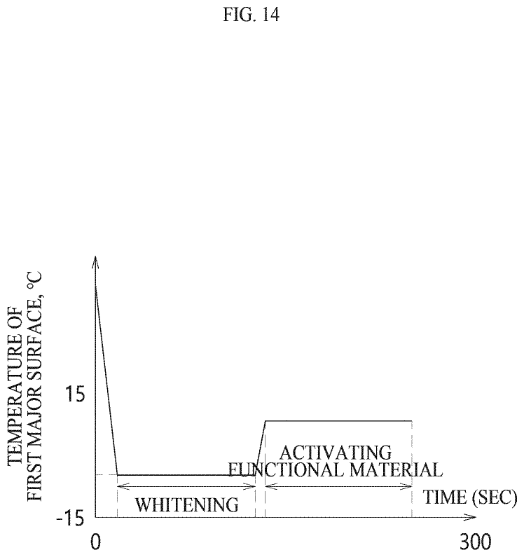

FIG. 14 is a graph showing temperature changes over time of a first major surface of a thermoelectric module according to the fourth embodiment of the present specification.

Because the activity of the functional material 21 accommodated in the contact layer 20 changes according to temperature, when the temperature of the first major surface 31 is within the activation temperature range of the functional material 21 accommodated in the contact layer 20, and thus the user's skin is cooled within the activation temperature range of the functional material 21, the skin improvement effect by the functional material 21 may increase.

Therefore, the controller 50 according to an embodiment of the present specification may control the thermoelectric module 30 to perform a functional material activation mode in which the first major surface 31 is cooled within the activation temperature range of the functional material 21 accommodated in the contact layer 20.

The controller 50 may control the thermoelectric module 30 to cool the first major surface 31 within the activation temperature range of the functional material 21 accommodated in the contact layer 20. For example, when the activation temperature range of the functional material 21 accommodated in the contact layer 20 ranges from 0° C. to 10° C., the controller 50 may control the thermoelectric module 30 to cool the first major surface 31 within the range of 0° C. to 10° C.

Also, the controller 50 may control the thermoelectric module 30 to cool the first major surface 31 at a temperature at which the activity is maximum within the activation temperature range of the functional material 21 accommodated in the contact layer 20. For example, when the temperature at which the activity is maximum within the activation temperature range of the functional material 21 accommodated in the contact layer 20 is 5° C., the controller 50 may control the thermoelectric module 30 to cool the first major surface 31 at 5° C.

Also, the controller 50 may control the thermoelectric module 30 to cool the first major surface 31 in the functional material activation at a temperature higher than the whitening temperatures of the whitening modes. This is to prevent damage to the user's skin while maximizing the effect of whitening the user's skin.

Also, the controller 50 may control the thermoelectric module 30 to cool the first major surface 31 in the functional material activation mode for a cooling interval longer than the whitening intervals of the whitening modes. This is to prevent damage to the user's skin while maximizing the effect of whitening the user's skin.

Also, the controller 50 may control the thermoelectric module 30 to perform at least one operation mode of the whitening modes and the functional material activation mode. For example, the controller 50 may control the thermoelectric module 30 to sequentially perform the whitening modes and the functional material activation mode in that order.

Also, the controller 50 may control the thermoelectric module 30 to perform the functional material activation mode simultaneously with the whitening mode during at least a portion of the whitening interval during which the whitening mode is performed. That is, the controller 50 may control the thermoelectric module 30 so that the whitening temperature range overlaps with the activation temperature range of the functional material 21 during at least a portion of the whitening interval during which the whitening mode is performed.

Also, the controller 50 may control the thermoelectric module 30 to perform the operation modes in various other combinations. For example, the controller 50 may control the thermoelectric module 30 to sequentially repeat the whitening modes and the functional material activation mode.

However, the fourth embodiment is not necessarily limited to the above-described method and may be practiced using various other methods, such as that in which the fourth embodiment is combined with other elements, according to circumstances.

### Fifth Embodiment

Hereinafter, a fifth embodiment in which the controller 50 controls the temperature of the first major surface 31 of the thermoelectric module 30 will be described. Descriptions overlapping those of the previous embodiments will be omitted. That is, descriptions of the technical ideas relating to the previous embodiments which may apply identically to the fifth embodiment will be omitted.

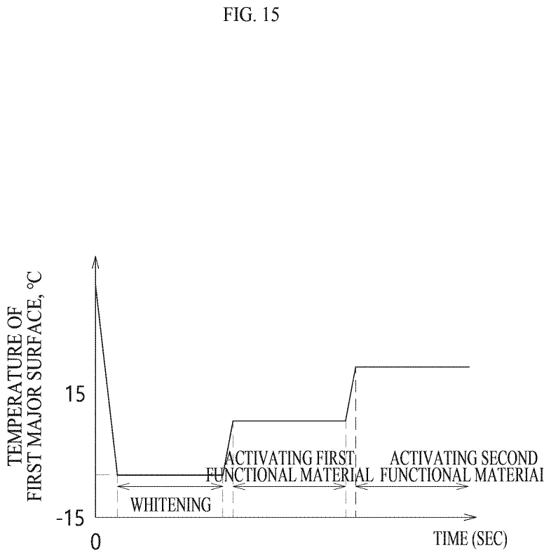

FIG. 15 is a graph showing temperature changes over time of a first major surface of a thermoelectric module according to a fifth embodiment of the present specification.

In order to obtain a comprehensive skin improvement effect, a plurality of functional materials 21 may be accommodated in the contact layer 20. In the case in which the plurality of functional materials 21 are accommodated in the contact layer 20, when a functional material activation mode is performed within an activation temperature range for activating any one functional material 21, another functional material may not be activated.

Therefore, when a plurality of functional materials 21 are accommodated in the contact layer 20, the controller 50 according to an embodiment of the present specification may control the thermoelectric module 30 to perform a first functional material activation mode, in which the first major surface 31 of the thermoelectric module 30 is cooled within a first activation temperature range of a first functional material 21-1 accommodated in the contact layer 20, and a second functional material activation mode, in which the first major surface 31 is cooled within a second activation temperature range of a second functional material 21-2 accommodated in the contact layer 20.

Referring to FIG. 15, the controller 50 may control the thermoelectric module 30 to perform the first functional material activation mode, in which the first major surface 31 of the thermoelectric module 30 is cooled within the first activation temperature range of the first functional material 21-1 accommodated in the contact layer 20, and the second functional material activation mode, in which the first major surface 31 is cooled within the second activation temperature range of the second functional material 21-2 accommodated in the contact layer 20.

Also, the controller 50 may control the thermoelectric module 30 to perform at least one operation mode of the whitening modes, the first functional material activation mode, and the second functional material activation mode. For example, the controller 50 may control the thermoelectric module 30 to sequentially perform the first functional material activation mode and the second functional material activation mode after performing the whitening modes.

Also, the controller 50 may control the thermoelectric module 30 to perform at least one of the first functional material activation mode and the second functional material activation mode simultaneously with the whitening mode during at least a portion of the whitening interval during which the whitening mode is performed. That is, the controller 50 may control the thermoelectric module 30 so that at least one of the first activation temperature range of the first functional material 21-1 and the second activation temperature range of the second functional material 21-2 overlaps the whitening temperature range during at least a portion of the whitening interval during which the whitening mode is performed.

Also, the controller 50 may control the thermoelectric module 30 to perform the operation modes in various other combinations. For example, the controller 50 may control the thermoelectric module 30 to sequentially repeat the whitening modes, the first functional material activation mode, and the second functional material activation mode.

However, the fifth embodiment is not necessarily limited to the above-described method and may be practiced using various other methods, such as that in which the fifth embodiment is combined with other elements, according to circumstances.

### Sixth Embodiment

Hereinafter, a sixth embodiment in which the controller 50 controls the temperature of the first major surface 31 of the thermoelectric module 30 will be described. Descriptions overlapping those of the previous embodiments will be omitted. That is, descriptions of the technical ideas relating to the previous embodiments which may apply identically to the sixth embodiment will be omitted.

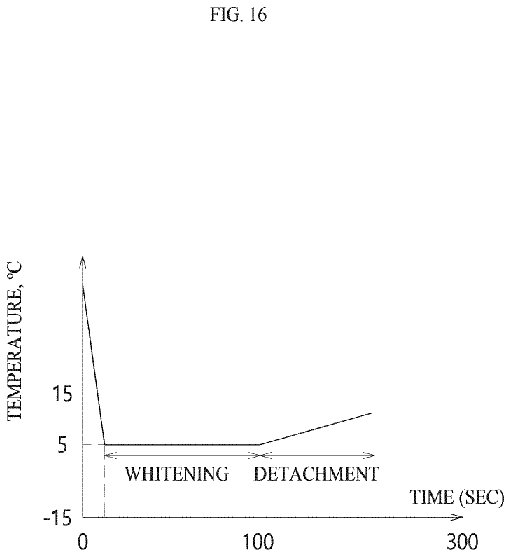

FIG. 16 is a graph showing temperature changes over time of a first major surface of a thermoelectric module according to a sixth embodiment of the present specification.

In a case in which, after the cooling of the first major surface 31 is completed by power applied to the thermoelectric module 30 by the controller 50, the power to the thermoelectric module 30 is cut off by the controller 50, the temperature of the thermoelectric module 30 rises due to heat generated from the second major surface 32 and rapidly approaches the high temperature of the second major surface 32. In this case, when the thermoelectric module 30 with an increased temperature suddenly applies heat to the user's skin which has been cooled to low temperature, the user may suddenly feel relatively uncomfortable due to hotness, and damage to the skin may occur due to the high temperature.

Therefore, the controller 50 according to an embodiment of the present specification may control the thermoelectric module 30 so that, after the cooling of the first major surface 31 is completed (e.g., the whitening mode, the functional material activation mode, or the like is completed), the temperature of the first major surface 31 does not increase rapidly before the user removes the mask. Referring to FIG. 16, it can be seen that, after the whitening mode in which the first major surface 31 is cooled at a cooling temperature of 5° C. for 100 seconds is completed, the thermoelectric module 30 is controlled by the controller 50 and performs a detachment mode in which the temperature of the first major surface 31 gradually increases. Hereinafter, the detachment mode refers to a mode in which, after the cooling of the first major surface 31 is completed (an operation mode is ended), the controller 50 controls the temperature of the first major surface 31.

In order to prevent the temperature of the first major surface 31 from rapidly increasing after the cooling of the first major surface 31 is completed, the controller 50 may control the thermoelectric module 30 to perform the detachment mode in which the temperature of the first major surface 31 gradually increases. For example, the controller 50 may control the thermoelectric module 30 so that, after the cooling of the first major surface 31 is completed, the temperature of the first major surface 31 increases by 0.3° C. per second.

Also, the controller 50 may control the thermoelectric module 30 to perform the detachment mode in which, after the cooling of the first major surface 31 is completed, the temperature of the first major surface 31 is maintained at a predetermined temperature. For example, the controller 50 may control the thermoelectric module 30 so that the temperature of the first major surface 31 gradually increases and reaches a temperature in a range of 5° C. to 30° C. Alternatively, the controller 50 may also control the thermoelectric module 30 so that, after the cooling of the first major surface 31 is completed, the temperature of the first major surface 31 is maintained as it is.

Also, the controller 50 may control the thermoelectric module 30 to maintain the detachment mode for a predetermined amount of time so that a sufficient amount of time is provided for the user to remove the mask 10 while the detachment mode is performed. For example, the controller 50 may control the thermoelectric module 30 to perform the detachment mode for an interval in a range of 10 seconds to 5 minutes.

Also, when the controller 50 obtains a signal indicating that the user has removed the mask 10, the controller 50 may control the thermoelectric module 30 to end the detachment mode. For example, the controller 50 may control the thermoelectric module 30 to end the detachment mode when a power off signal is received from the user or a signal indicating that the user's skin is not detected is obtained via a touch sensing module.

Also, the controller 50 may control an output module to output a notification when the detachment mode starts or ends. For example, the controller 50 may control the output module to output vibration or a voice message that notifies of detachment of the mask upon the start of the detachment mode. Alternatively, the controller 50 may control a vibration generating module to output vibration when the detachment mode starts or ends.

However, the sixth embodiment is not necessarily limited to the above-described method and may be practiced using various other methods, such as that in which the sixth embodiment is combined with other elements, according to circumstances.

### Additional Embodiments

Hereinafter, various additional embodiments in which the controller 50 controls the mask 10 will be described. Descriptions overlapping those of the previous embodiments will be omitted. That is, descriptions of the technical ideas relating to the previous embodiments which may apply identically to the additional embodiments will be omitted.

Because cooling conditions for achieving the optimum whitening effect are different for each individual, the mask 10 may cool the user's skin under different cooling conditions for each individual in order to achieve the optimum whitening effect.

Therefore, the controller 50 according to an embodiment of the present specification may control the thermoelectric module 30 to perform an operation mode set by the user other than a preset operation mode. Alternatively, the controller 50 may control the thermoelectric module 30 to perform an operation mode that results from arbitrarily modifying a preset operation mode.

The controller 50 may control the thermoelectric module 30 to cool the first major surface 31 under a cooling condition received from the input module 52. For example, the controller 50 may control the thermoelectric module 30 to cool the first major surface 31 according to data relating to cooling conditions of the first major surface 31 (the cooling interval of the first major surface 31, the cooling temperature of the first major surface 31, cooling order, cooling area, and the like) which are received from the input module 52.

Also, the controller 50 may control the thermoelectric module 30 to cool the first major surface 31 according to a cooling condition obtained from an external device via the communication module 51. For example, the controller 50 may control the thermoelectric module 30 to cool the first major surface 31 according to data relating to cooling conditions of the first major surface 31 (the cooling interval of the first major surface 31, the cooling temperature of the first major surface 31, cooling order, cooling area, and the like) which are received from an external device via the communication module 51.

Embodiments have been described above with reference to the accompanying drawings, but those of ordinary skill in the art may make various modifications and changes from the description above. For example, appropriate results can be achieved even when the described techniques are performed in a different order from the described methods or when the conditions are different.

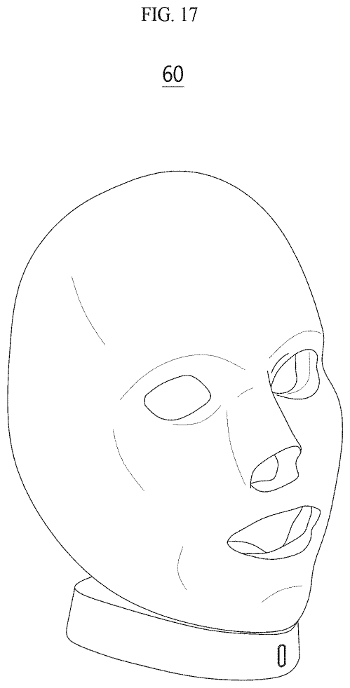

FIG. 17 illustrates a cradle on which a mask is mounted according to an embodiment of the present specification.

Referring to FIG. 17, a cradle 60 for keeping the mask 10 while it is not in use may be provided in the shape corresponding to the mask 10. Of course, the cradle 60 is not limited to the present embodiment and may be provided in various other shapes on which the mask 10 may be mounted. For example, as illustrated in FIG. 17, the cradle 60 may be provided in the form in which, when the mask 10 is mounted, the contact layer 20 comes in contact with one surface of the cradle 60. However, the cradle 60 may also be provided in the form in which, when the mask 10 is mounted, the heat dissipation layer 40 comes in contact with one surface of the cradle 60.

Also, the cradle 60 may include a heat dissipation means (not illustrated) such as a separate thermoelectric module, fan, fluid circulator or the like for aiding in heat dissipation by the heat dissipation layer 40 when the mask 10 is mounted on the cradle 60.

According to an embodiment, when the mask 10 is mounted on the cradle 60 which is provided in the form in which, when the mask 10 is mounted, the contact layer 20 comes in contact with one surface of the cradle 60, a direction of current applied to the thermoelectric module 30 of the mask 10 is reversed, and the mask 10 may cool the heat dissipation layer 40 through the second major surface 32, and the cradle 60 may dissipate, via a separate heat dissipation means, heat generated from the first major surface 31 of the thermoelectric module 30. For example, when the mask 10 is mounted on the cradle 60, the mask 10 may, via the second major surface 32, cool the heat dissipation layer 40 below a melting point of a phase-change material included in the heat dissipation layer 40, and the cradle 60 may, via a separate fan or thermoelectric module, dissipate heat generated from the first major surface 31.

According to another embodiment, when the mask 10 is mounted on the cradle 60 which is provided in the form in which, when the mask 10 is mounted, the heat dissipation layer 40 comes in contact with one surface of the cradle 60, the cradle 60 may cool the heat dissipation layer 40 via a separate heat dissipation means. For example, when the mask 10 is mounted on the cradle 60, the cradle 60 may, via a thermoelectric module disposed at one surface of the cradle 60, cool the heat dissipation layer 40 below a melting point of a phase-change material included in the heat dissipation layer 40.

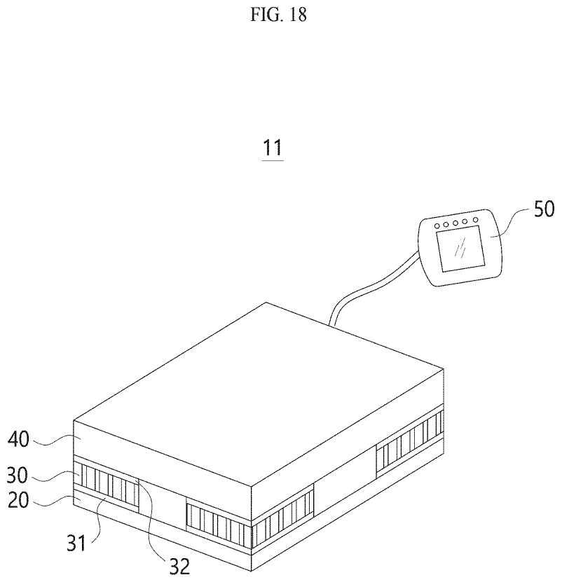

FIG. 18 is a perspective view of a device for whitening skin according to an embodiment of the present specification.

Referring to FIG. 18, a device for whitening skin 11 may have a basic form for being adhered to various body parts of a user and be provided in the form of a pad. FIG. 18 only illustrates the device for whitening skin 11 provided in the form of a pad, but the device for whitening skin 11 may be provided in various forms. For example, the device for whitening skin 11 may be provided in forms corresponding to various body parts in order to be adhered to various body parts of the user and evenly cool the user's skin. That is, the device for whitening skin 11 may be provided to be flexible and bendable.

Also, the device for whitening skin 11 may include a contact layer 20, at least one thermoelectric module 30, a heat dissipation layer 40, and a controller 50.

Because the contact layer 20, the thermoelectric module 30, the heat dissipation layer 40, and the controller 50 have been described above, detailed descriptions thereof will be omitted.

Meanwhile, although not illustrated in the drawings, the device for whitening skin 11 according to an embodiment of the present specification may further include a strap, a band, or the like that provides tension so that, upon use of the device for whitening skin 11 by the user, a surface of the device for whitening skin 11 that comes in contact with the user's skin is sufficiently adhered thereto.

However, the device for whitening skin 11 is not limited by the above-described configuration. The device for whitening skin 11 may include elements more or less than those described above, and each element is not limited by the above description.

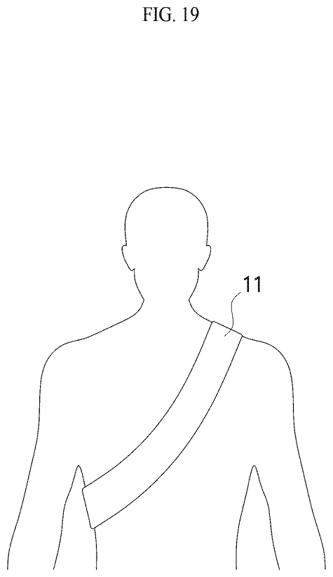

FIG. 19 is a view illustrating a state in which a device for whitening skin is used for an upper body part of a user according to an embodiment of the present specification.

Referring to FIG. 19, the device for whitening skin 11 may be provided in the form of a pad and used for the upper body part of the user.

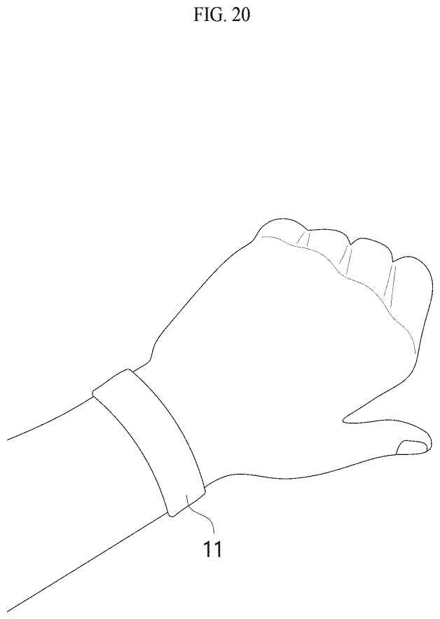

FIG. 20 is a view illustrating a state in which a device for whitening skin is used for a wrist area of a user according to an embodiment of the present specification.

Referring to FIG. 20, the device for whitening skin 11 may be provided in the form of a band corresponding to a wrist area and used for the wrist area of the user.

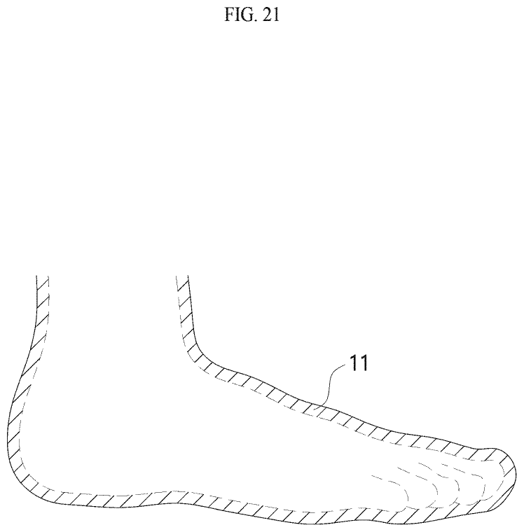

FIG. 21 is a view illustrating a state in which a device for whitening skin is used for a foot area of a user according to an embodiment of the present specification.

Referring to FIG. 21, the device for whitening skin 11 may be provided in the shape corresponding to a foot area and used for the foot area of the user.

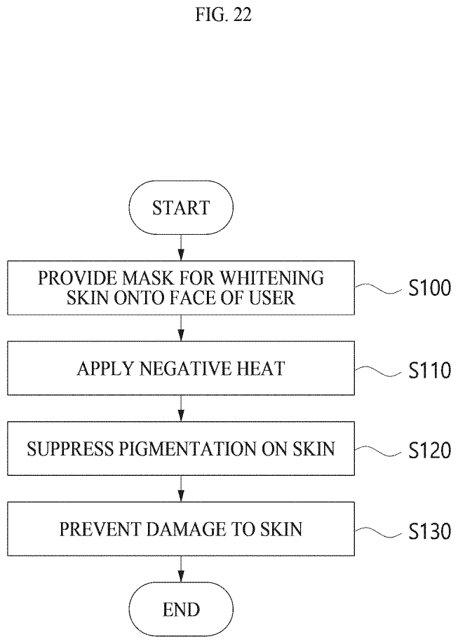

FIG. 22 is a flowchart of a method for whitening skin using a mask for whitening skin according to an embodiment of the present specification.

Referring to FIG. 22, a method for whitening skin using the mask for whitening skin 10 may include positioning the mask for whitening skin 10 onto the face of a user (S100), applying negative heat (S110), suppressing pigmentation of the skin (S120), and preventing damage to the facial skin (S130).

The mask for whitening skin 10 may be positioned on the face of the user so that a lower portion of a contact layer 20 adheres to the face (S100).

For example, the mask for whitening skin 10 may be positioned on the face of the user so that the lower portion of the contact layer 20 adheres to the face. wherein the mask for whitening skin 10 includes the contact layer 20 which has the lower portion coming in contact with the face of the user and is provided with a flexible material, at least one thermoelectric module 30 which has two major surfaces 31 and 32 including a first major surface 31 disposed in contact with an upper portion of the contact layer 20 and a second major surface 32 positioned opposite to the first major surface 31 and, as power is applied, cools the first major surface 31, a heat dissipation layer 40 disposed at an upper portion of the thermoelectric module 30 so as to come in contact with the second major surface 32 of the thermoelectric module 30 and configured to receive, via the second major surface 32, heat generated upon cooling of the first major surface 31 and dissipate the received heat, and a controller 50 configured to control the thermoelectric module 30.

Also, the mask for whitening skin 10 may further include a strap, a band, or the like that provides tension so that a surface of the mask for whitening skin 10 that comes in contact with the user's skin is sufficiently adhered thereto.

Also, the mask for whitening skin 10 may apply negative heat to the user's skin via the thermoelectric module 30 (S110).

The mask for whitening skin 10 may perform skin whitening via the thermoelectric module 30 and apply negative heat to the user's skin to prevent damage to the skin. For example, the mask for whitening skin 10 may, as power is applied to the thermoelectric module 30, cool the first major surface 31 and apply negative heat to the user's skin via the contact layer 20.

Also, the mask for whitening skin 10 may suppress pigmentation by melanocytes on the skin (S120).

The mask for whitening skin 10 may, via the thermoelectric module 30, apply negative heat to the user's skin at a temperature at which pigmentation by melanocytes may be suppressed.

For example, the mask for whitening skin 10 may cool the first major surface 31 at a cooling temperature such that the skin of the user reaches a target temperature at which pigmentation by melanocytes is suppressed, wherein the target temperature is in a range of 4° C. to 27° C. and the cooling temperature is in a range of −15° C. to 15° C. and lower than the target temperature.

Also, the mask for whitening skin 10 may prevent damage to the skin (S130).

The mask for whitening skin 10 may, via the thermoelectric module 30, apply negative heat to the user's skin for an interval during which damage to the skin does not occur.

For example, the mask for whitening skin 10 may prevent damage to the skin by maintaining a cooling interval, during which the cooling of the first major surface 31 occurs, in a range of 5 seconds, which is necessary for suppressing the pigmentation, to less than 300 seconds, at which damage to the skin begins.

Because the descriptions given above may apply identically to the method for whitening skin illustrated in FIG. 22, more detailed descriptions will be omitted.

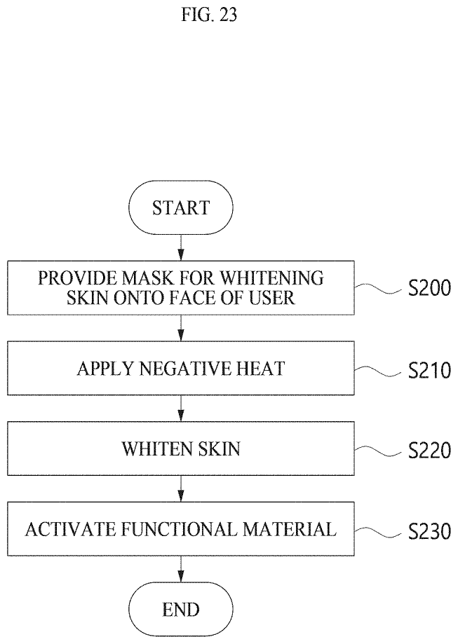

FIG. 23 is a flowchart of a method for whitening skin using a mask for whitening skin according to an embodiment of the present specification.

Referring to FIG. 23, a method for whitening skin using a mask for whitening skin 10 may include positioning the mask for whitening skin 10 on the face of a user (S200), applying negative heat (S210), whitening the skin (S220), and activating a functional material (S230).

The mask for whitening skin 10 may be positioned on the face of the user so that a lower portion of a contact layer 20 adheres to the face (S200).

For example, the mask for whitening skin 10 may be positioned on the face of the user so that the lower portion of the contact layer 20 adheres to the face, wherein the mask for whitening skin 10 includes the contact layer 20 which has the lower portion coming in contact with the face of the user and is provided with a flexible material, a functional material 21 which is accommodated in the contact layer 20, is configured to improve the skin of the face of the user, and has an activation temperature range, at least one thermoelectric module 30 which has two major surfaces 31 and 32 including a first major surface 31 disposed in contact with an upper portion of the contact layer 20 and a second major surface 32 positioned opposite to the first major surface 31 and, as power is applied, cools the first major surface 31, a heat dissipation layer 40 disposed at an upper portion of the thermoelectric module 30 so as to come in contact with the second major surface 32 of the thermoelectric module 30 and configured to receive, via the second major surface 32, heat generated upon cooling of the first major surface 31 and dissipate the received heat, and a controller 50 configured to control the thermoelectric module 30.

Also, the mask for whitening skin 10 may apply negative heat to the user's skin via the thermoelectric module 30 (S210).

The mask for whitening skin 10 may perform skin whitening via the thermoelectric module 30 and apply negative heat to the user's skin to activate the functional material 21. For example, the mask for whitening skin 10 may, as power is applied to the thermoelectric module 30, cool the first major surface 31 and apply negative heat to the user's skin via the contact layer 20.

Also, the mask for whitening skin 10 may perform skin whitening by applying negative heat to the user's skin (S220).

The mask for whitening skin 10 may perform skin whitening by, via the controller 50, cooling the first major surface 31 within a whitening temperature range in which pigmentation by melanocytes is suppressed.

For example, the mask for whitening skin 10 may cool the first major surface 31 at a cooling temperature such that the skin of the user reaches a target temperature at which pigmentation by melanocytes is suppressed, wherein the target temperature is in a range of 4° C. to 27° C. and the cooling temperature is in a range (whitening temperature range) of −15° C. to 15° C. and lower than the target temperature, and perform skin whitening by maintaining a cooling interval (whitening interval), during which the cooling of the first major surface 31 occurs, in a range of 5 seconds, which is necessary for suppressing the pigmentation, to less than 300 seconds, at which damage to the skin begins.

Also, the mask for whitening skin 10 may apply negative heat to the user's skin and activate the functional material 21 (S230).

In order to increase the activity of the functional material 21 accommodated in the contact layer 20, the mask for whitening skin 10 may, via the thermoelectric module 30, apply negative heat within the activation temperature range of the functional material 21.

For example, the mask for whitening skin 10 may activate the functional material 21 by, via the controller 50, causing the whitening temperature range and the activation temperature range of the functional material 21 to overlap during at least a portion of the whitening interval, during which the cooling of the first major surface occurs within the whitening temperature range.

Because the descriptions given above may apply identically to the method for whitening skin illustrated in FIG. 23, more detailed descriptions will be omitted.

On the other hand, the above-described method for whitening skin may be performed in the same way using the device for whitening skin 11 instead of the mask for whitening skin 10. For example, the device for whitening skin 11 may not only whiten the user's facial skin but also whiten the skin on other body parts of the user by being positioned on a hand area, a foot area, an upper body part, or the like of the user.

Hereinafter, the heat dissipation layer 40 which receives heat generated from the thermoelectric module 30 and dissipates the received heat using a method of exchanging heat through fluid circulation according to an embodiment of the present specification will be described. For example, the mask 10 may include a fluid circulation unit 80 configured to receive heat generated from the thermoelectric module 30 and dissipate the received heat.

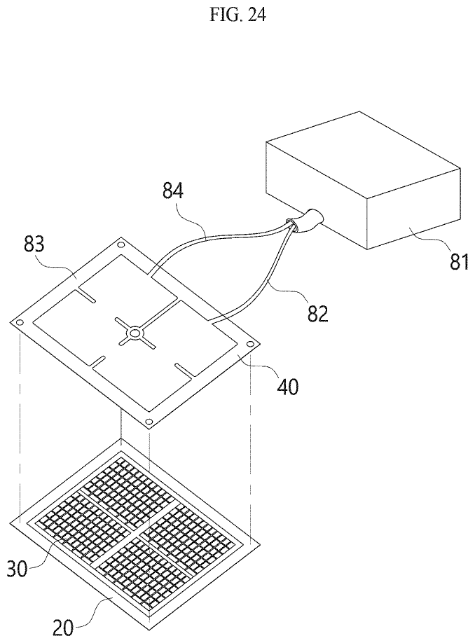

FIG. 24 illustrates a schematic exploded perspective view of a mask including a fluid circulation unit according to an embodiment of the present specification. For convenience, the mask 10 including the fluid circulation unit 80 has been illustrated as having a rectangular shape but may also be manufactured in a shape corresponding to a face.

The fluid circulation unit 80 is an element that maintains a predetermined temperature difference between both major surfaces 31 and 32 of the thermoelectric module 30 by removing heat generated from the thermoelectric module 30. The fluid circulation unit 80 may, via a fluid inlet passage 82, supply a circulating fluid to a fluid chamber 83 which has one surface coming in contact with the thermoelectric module 30, exchange heat with the thermoelectric module 30 in the fluid chamber 83, and then retrieve the circulating fluid discharged via a fluid outlet passage 84, thereby circulating the circulating fluid.

To this end, the fluid circulation unit 80 may include a fluid supply part 81, the fluid inlet passage 82, the fluid chamber 83, and the fluid outlet passage 84.

The fluid supply part 81 may supply a circulating fluid, which has a predetermined temperature for receiving heat generated from the thermoelectric module 30 and dissipating the received heat, to the fluid chamber 83. To this end, the fluid supply part 81 may include a fluid tank 85, a pump 86, and a cooler 87.

The fluid tank 85 may store therein a predetermined amount of circulating fluid which will be supplied to the fluid chamber 83. Also, the fluid tank 85 may receive the circulating fluid retrieved via the fluid outlet passage 84 and store the retrieved circulating

The pump 86 may be connected to the fluid tank 85 and, by pumping, supply the circulating fluid stored in the fluid tank 85 to the fluid chamber 83 via the fluid inlet passage 82. Also, by pumping, the pump 86 may retrieve, via the fluid outlet passage 84, the circulating fluid which finished exchanging heat in the fluid chamber 83 that comes in contact with one surface of the thermoelectric module 30.

The cooler 87 may release heat absorbed by the thermoelectric module 30 to the outside. The cooler 87 may cool the circulating fluid which finished exchanging heat that is retrieved via the fluid outlet passage 84. For example, the cooler 87 may include a radiator, a chiller, and the like for cooling a fluid which will be supplied to the fluid chamber 83.

Also, the cooler 87 may further include a fan.

The fluid inlet passage 82 and the fluid outlet passage 84 may be connected to the fluid supply part 81 and serve as passages that allow the circulating fluid for cooling to pass from the fluid tank 85 to the fluid chamber 83.

Also, the fluid inlet passage 82 and the fluid outlet passage 84 may be connected to the fluid chamber 83. For example, the fluid inlet passage 82 may be connected to a coolant inlet that supplies coolant formed in the fluid chamber 83. Also, the fluid outlet passage 84 may be connected to a coolant outlet that discharges the coolant formed in the fluid chamber 83.

The fluid chamber 83 may be disposed so that at least one surface comes in contact with the thermoelectric module 30. For example, the fluid chamber 83 may be adhered to at least one surface of the thermoelectric module 30 by an adhesive or silicone having high thermal conductivity.

Also, a fluid that absorbs heat generated from the thermoelectric module 30 may pass through the fluid chamber 83, and the fluid chamber 83 may exchange heat with one surface 32 of the thermoelectric module 30 by the fluid passing through the fluid chamber 83. The fluid chamber 83 may allow heat generated from the thermoelectric module 30 to be transferred to the fluid that is supplied via the fluid inlet passage 82 and passes through the fluid chamber 83 and may allow the fluid, to which heat is transferred, to be discharged to the fluid tank 85 via the fluid outlet passage 84.

Also, the fluid chamber 83 may be formed of a material with high thermal conductivity to improve the efficiency of transferring heat generated from the thermoelectric module 30 to the fluid inside the fluid chamber 83. For example, the fluid chamber 83 may be formed of aluminum or the like.

The fluid chamber 83 may also be formed of a material with high flexibility to secure high flexibility of the mask 10. For example, the fluid chamber 83 may be formed of plastic or the like that has high thermal conductivity and flexibility.

According to an embodiment of the present specification, in order to secure high flexibility of the mask 10, the fluid chamber 83 may be provided to be segmented into a plurality of chambers 83 of small sizes. The plurality of fluid chambers 83 may be connected to each other via a connection line (hose). The circulating fluid supplied from the fluid supply part 81 to one fluid chamber 83 may be provided to the plurality of chambers 83 via the connection line (hose). Alternatively, the plurality of fluid chambers 83 may separately receive the circulating fluid from the fluid supply part 81 via the fluid inlet passage 82 and the fluid outlet passage 84 which are separate.

The plurality of fluid chambers 83 may be connected to each other in series and/or in parallel. Each chamber 83 may separately receive the circulating fluid from the fluid supply part 81 via the fluid inlet passage 82 and the fluid outlet passage 84 which are separate. Alternatively, one chamber 83 may receive the circulating fluid from the fluid supply part 81 via one fluid inlet passage 82 and one fluid outlet passage 84, and the received circulating fluid may be provided to other chambers 83 via the connection line (hose).

One of the plurality of fluid chambers 83 may be disposed to come in contact with the at least one thermoelectric module 30. One fluid chamber 83 may be disposed to come in contact with at least one surface of the at least one thermoelectric module 30, and thus a separate module formed of one fluid chamber 83 and at least one thermoelectric module 30 may be provided, For example, one fluid chamber 83 may be disposed to come in contact with at least one surface of one thermoelectric module 30, and a separate module formed of one fluid chamber 83 and one thermoelectric module 30 may be provided.

The controller 50 may control the overall operation of the fluid circulation unit 80. For example, when a measured temperature of the second major surface 32 of the thermoelectric module 30 is higher than a predetermined temperature, the controller 50 may increase the amount of fluid circulating inside the fluid chamber 83 or lower a temperature at which the fluid is supplied. As another example, the controller 50 may determine whether to operate the fluid circulation unit 80 corresponding to the on/off of power of the thermoelectric module 30.

The fluid circulation unit 80 is not necessarily controlled only by the controller 50 and may also be controlled by a separate controller (not illustrated).

Also, the fluid circulation unit 80 may receive power via a separate power supply part (not illustrated) or receive power from a battery (not illustrated) separately accommodated in the fluid supply part 81.

Also, the fluid circulation unit 80 may be implemented as an element included in the cradle 60. For example, the cradle 60 may include the fluid supply part 81, dissipate heat generated from the thermoelectric module 30 of the mask 10, and serve as a place where the mask 10 is mounted.

The circulating fluid may be a material with high thermal efficiency, e.g., high specific heat, for exchanging heat or may be a material which is non-conductive and has low reactivity with the thermoelectric module 30. Specifically, the circulating fluid may be an oil-based fluid, and examples thereof may include natural oil, mineral oil, synthetic oil (e.g., silicone oil), non-conductive liquids (single-phase cooling fluids: 3M's FC-770, FC-3283, FC-40, FC-43, FC-70, FC-72, FC-84, FC-87, etc./two-phase cooling fluids: 3M's Novec Engineered fluids), and the like. Of course, other kinds of fluids such as water may also be used as the circulating fluid, and the circulating fluid may be various kinds of fluids used for exchanging heat.

However, the fluid circulation unit 80 is not limited to the above. The fluid circulation unit 80 may be provided as more or less fluid circulation units 80, and each fluid circulation unit 80 is not limited by e above description.

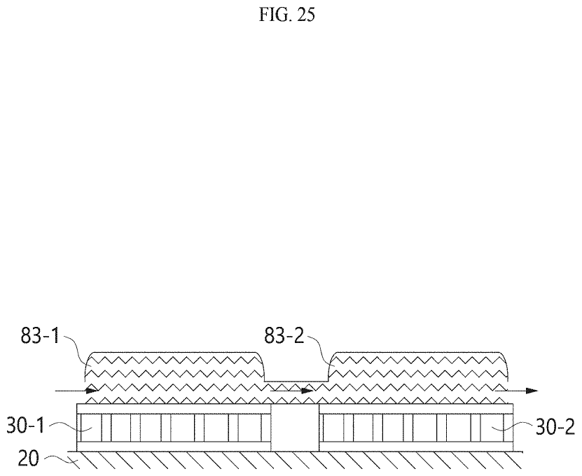

FIG. 25 illustrates a cross-sectional view of the mask including the fluid circulation unit according to an embodiment of the present specification.

Referring to FIG. 25, a plurality of fluid chambers 83-1 and 83-2 may be disposed to come in contact with at least one surface of a thermoelectric module 30-1 and at least one surface of a thermoelectric module 30-2, respectively.

When a user operates an operation switch (not illustrated) via the controller 50 while the contact layer 20 is attached to the face of the user, from the operation of the operation switch, the controller 50 may drive the pump 86 while supplying power to the thermoelectric module 30 via a power line, and the pump 86 may control the fluid circulation unit 80 to supply the fluid stored in the fluid tank 85 to the fluid chamber 83 via the fluid inlet passage 82.

The circulating fluid may circulate inside the plurality of fluid chambers 83-1 and 83-2 and be retrieved into the fluid tank 85 via the fluid outlet passage 84. Accordingly, the mask 10 may dissipate heat generated from the thermoelectric module 30 by the fluid circulating inside the fluid chamber 83.

Although FIG. 25 illustrates the plurality of fluid chambers 83-1 and 83-2 as being connected, one fluid chamber 83-1 may form a separate module with one thermoelectric module 30-1 and separately receive a fluid from the fluid supply part 81.

Also, in the method for whitening skin according to the embodiments of the present specification described above, steps in which a subject performing the step is not particularly specified may be performed by the controller 50.

According to an embodiment of the present specification, by transferring negative heat, which is generated according to an endothermic reaction of a thermoelectric module, without change to a face, it is possible to conveniently and effectively cool facial skin of a user and obtain a whitening effect.

According to another embodiment of the present specification, by controlling a cooling temperature of a cooling surface of a thermoelectric module and an interval during which cooling occurs, it is possible to obtain an effect of preventing damage to the skin.

According to another embodiment of the present specification, by maintaining a heat generating surface of a thermoelectric module at a predetermined temperature, it is possible to obtain an effect of allowing smooth operation of the thermoelectric module.

According to another embodiment of the present specification, by controlling a temperature of a thermoelectric module to be within a temperature range in which a functional material is active, it is possible to obtain an effect of activating the functional material.

According to another embodiment of the present specification, by controlling a temperature of a thermoelectric module, it is possible to obtain additional skin improvement effects such as lipolysis and reduction of swelling.

Advantageous effects of the present disclosure are not limited to those described above, and other unmentioned advantageous effects should be clearly understood by those of ordinary skill in the art to which the present invention pertains from the present specification and the accompanying drawings.

A method according to an embodiment may be implemented in the form of a program command that can be executed through various computer means and may be recorded in a computer-readable medium. The computer-readable medium may include program commands, data files, data structures, and the like alone or in combination. The program commands recorded in the medium may be those specially designed and configured for the embodiment or those known to those skilled in the art of computer software and usable. Examples of the computer-readable recording medium include magnetic media such as a hard disk, a floppy disk, and a magnetic tape, optical media such as compact disk-read only memory (CD-ROM) and a digital versatile disk (DVD), magneto-optical media such as a floptical disk, and hardware devices such as a read only memory (ROM), a random access memory (RAM), and a flash memory specially configured to store and execute a program command. Examples of the program command include high-level language codes that may be executed by a computer using an interpreter or the like as well as machine language codes generated by a compiler. The above-mentioned hardware device may be configured to operate as one or more software modules to execute operations according to an embodiment, and vice versa.

The embodiments have been described above with reference to the accompanying drawings, but those of ordinary skill in the art may make various modifications and changes from the description above. For example, appropriate results can be achieved even when the described techniques are performed in a different order from the described methods and/or when elements such as systems, structures, devices, and circuits are coupled or combined in a different form from described methods or replaced or substituted with other elements or their equivalents.

Therefore, other implementations, other embodiments, and equivalents to the claims are within the scope of the claims below.

1. A skincare facial mask comprising:
a first mask layer configured to contact a face when the skincare facial mask is worn over the face, wherein the first mask layer comprises a first set of cutouts configured to be aligned with two eves, a nose and a mouth of the face when the skincare facial mask is worn over the face;
a second mask layer overlaying the first mask layer such that the first mask layer is interposed between the face and the second mask layer when the skincare facial mask is worn over the face, wherein the second mask layer comprises a second set of cutouts corresponding to the first set of cutouts of the first mask layer such that the second set of cutouts of the second mask layer are configured to be aligned with the two eyes, nose and mouth of the face when the skincare facial mask is worn over the face;
at least one skincare material embedded in the first mask layer such that the at least one skincare material is configured to contact the face when the skincare facial mask is worn over the face;
at least one thermoelectric module interposed between the first mask layer and the second mask layer, wherein the at least one thermoelectric module comprises a heat-absorbing surface facing and contacting the first mask layer and a heat-releasing surface facing the second mask layer such that the at least one thermoelectric module is configured to cool the first mask layer, which in turn is to cool at least one area of the face when the skincare facial mask is worn over the face;
a fluid circulation system comprising at least one fluid chamber and a fluid tank, wherein the at least one fluid chamber is interposed between the first mask layer and the second mask layer, wherein the at least one fluid chamber is in fluid communication with the fluid tank for circulating fluid between the at least one fluid chamber and the fluid tank, wherein the at least one fluid chamber is in contact with the heat-releasing surface of the at least one thermoelectric module for taking heat from the heat-releasing surface of the at least one thermoelectric module; and
at least one controller configured to control the at least one thermoelectric module to absorb heat from the first mask layer through the heat-absorbing surface and also to control the fluid circulation system to absorb heat from the at least one thermoelectric module through the heat-releasing surface such that the at least one skincare material embedded in the first mask layer is cooled to a temperature within a predetermined temperature range and is maintained at a temperature within the predetermined temperature range for a predetermine period.

2. The skincare facial mask of claim 1, further comprising a skin temperature sensor configured to measure a skin temperature of the face,
wherein the at least one controller is configured to control the at least one thermoelectric module based on the skin temperature measured with the skin temperature sensor.

3. The skincare facial mask of claim 2, wherein the at least one controller is configured to control the at least one thermoelectric module to maintain the skin temperature at a target temperature for a period of 4 seconds to 120 seconds.

4. The skincare facial mask of claim 1, further comprising:
a vibration generator disposed at least one of the first mask layer and the second mask layer and configured to generate vibration for applying to the face.

5. The skincare facial mask of claim 1, further comprising:
a touch sensor configured to detect a contact of the skincare facial mask to the face, wherein the at least one controller is configured to control the at least one thermoelectric module to start cooling of the heat-absorbing surface when the contact of the face is detected by the touch sensor.

6. (canceled)

7. The skincare facial mask of claim 1, wherein the at least one thermoelectric module comprises a plurality of thermoelectric modules, and
wherein the at least one controller is configured to control each of the plurality of thermoelectric modules.

8. The skincare facial mask of claim 7, wherein the plurality of thermoelectric modules corresponds to a plurality of face areas defined based on a skin temperature profile.

9. The skincare facial mask of claim 7, wherein the plurality of thermoelectric modules comprises a first thermoelectric module corresponding to a first section of the heat-absorbing surface and a second thermoelectric module corresponding to a second section of the heat-absorbing surface, and
wherein the at least one controller is configured to control the first thermoelectric module to cool the first section for a first cooling interval and further configured to control the second thermoelectric module to cool the second section for a second cooling interval, the first cooling interval being greater than the second cooling interval.

10. The skincare facial mask of claim 7, wherein the plurality of thermoelectric modules comprises include a first thermoelectric module corresponding to a first section of the heat-absorbing surface and a second thermoelectric module corresponding to a second section of the heat-absorbing surface, and
wherein the at least one controller is configured to control the first thermoelectric module to cool the first section at a first cooling temperature and further configured to control the second thermoelectric module to cool the second section at a second cooling temperature, the first cooling temperature being higher than the second cooling temperature.

11. The skincare facial mask of claim 1, wherein the at least one controller further comprises an input module configured to obtain a cooling condition of the heat-absorbing surface, and wherein the at least one controller is configured to control the at least one thermoelectric module to cool the heat-absorbing surface according to the cooling condition inputted from the input module.

12. The skincare facial mask of claim 1, wherein the at least one controller further comprises a communication module configured to communicate with an external device, and wherein the at least one controller is configured to control the at least one thermoelectric module to cool the heat-absorbing surface according to a cooling condition obtained from the communication module.

13. The skincare facial mask of claim 1, wherein the at least one controller is configured to control the at least one thermoelectric module to cool the heat-absorbing surface according to a first cooling condition related to a swelling reduction effect or a second cooling condition related to lipolysis effect during a cooling of the heat-absorbing surface.

14. (canceled)

15. (canceled)

16. (canceled)

17. (canceled)

18. (canceled)

19. The skincare facial mask of claim 1, wherein the at least one skincare material comprises at least one selected from the group consisting of resorcinol, hexyl resorcinol, butyl resorcinol, phenyl ethyl resorcinol, resorcinol acetate, other resorcinol derivatives, niacinamide, magnesium ascorbylphosphate, ascorbyl glucoside, ascorbyl tetraisopalmitate, ascorbyl dipalmitate, arbutin, α-bisabolol, ethyl ascorbyl ether, polyphenol derivatives, L-glutathione, tranexamic acid, 4-methoxysalicylic acid potassium salt (KCl) derivatives, glycyrrhizine, azelaic acid, azelaic acid derivatives, azeloyl diglycine, nicotinamide, nicotinamide derivatives, resveratrol, resveratrol derivatives, glycyrrhiza flavonoids, ellagic acid, papain, mandelic acid, mandelic acid derivatives, heptapeptide-1, kojic acid, kojic acid derivatives, jasmine extract, mulberry extract, paper mulberry extract, licorice extract, ginseng extract, salvia miltiorrhiza extract, corn extract, chrysanthemum extract, bark root extract, thyme extract, white fresh root extract, polygon extract, magnolia tree extract, angelica root extract, phyllanthus emblica (fruit) extract, and citrus extract.
